(19) United States
(12) Patent Application Publication
(10) Pub. No.: US 2021/0169984 A1
(43) Pub. Date: Jun. 10, 2021

(54) SENSITIZING CANCER TO DEATH RECEPTOR AGONISTS WITH KINASE INHIBITORS

(71) Applicant: The Johns Hopkins University, Baltimore, MD (US)

(72) Inventors: Seulki Lee, Elkridge, MD (US); Martin Pomper, Baltimore, MD (US); Yumin Oh, Elkridge, MD (US); Magdalena Scully, Mount Airy, MD (US)

(21) Appl. No.: 16/071,116

(22) PCT Filed: Jan. 19, 2017
(86) PCT No.: PCT/US17/14051

§ 371 Date: Jul. 19, 2018

Related U.S. Application Data
(60) Provisional application No. 62/280,222, filed on Jan. 19, 2016.

(51) Int. Cl. A61K 38/19; A61K 9/00; A61K 9/20; A61K 47/60; A61K 31/4709; A61K 31/506; A61K 31/517; A61K 31/496; A61K 31/44; A61K 31/352; A61K 31/277; A61K 31/52; A61K 31/4745; A61K 31/4706; A61K 31/5377; A61K 31/454; A61K 31/4465; A61K 31/437; A61K 31/4439; A61K 31/519; A61K 31/453; A61K 31/194; A61K 31/444; A61K 31/404; A61K 31/53; A61K 31/4985; A61K 31/5025; A61P 35/00; G01N 33/50

(52) U.S. Cl. CPC A61K 38/191; G01N 2333/912; A61K 9/20; A61K 47/60; A61K 31/4709; A61K 31/506; A61K 31/517; A61K 31/496; A61K 31/44; A61K 31/352; A61K 31/277; A61K 31/52; A61K 31/4745; A61K 31/4706; A61K 31/5377; A61K 31/454; A61K 31/4465; A61K 31/437; A61K 31/4439; A61K 31/519; A61K 31/453; A61K 31/194; A61K 31/444; A61K 31/404; A61K 31/53; A61K 31/4985; A61K 31/5025; A61P 35/00; G01N 33/5011; A61K 9/0053

(57) ABSTRACT

The present invention relates to methods for sensitizing cancers to death receptor agonist therapies, comprising use of one or more kinase inhibitors. Such kinase inhibitors can be administered and identified as effective for sensitizing to death receptor agonist therapy in vitro or in vivo. Screening for additional kinase inhibitors and other agents that sensitize cancer cells to death receptor agonists is also contemplated, as is preventing and/or treatment of cancer using combination therapies that include such agents (e.g., kinase inhibitor(s) and death receptor agonist(s)). Formulations and kits including such combined agents are also contemplated and described, as are diagnostic/imaging agents that allow for advanced tracking of therapeutic outcome.

### RELATED APPLICATIONS

This application claims the benefit of priority under 35 U.S.C. § 119(e) to U.S. Provisional Application No: 62/280,222, filed Jan. 19, 2016, which is incorporated herein by reference in its entirety.

### STATEMENT REGARDING FEDERALLY SPONSORED RESEARCH OR DEVELOPMENT

This invention was made with government support under ROOEB013450 awarded by the National Institutes of Health (NIH) and the National Institute for Biomedical Imaging and Bioengineering (NIBIB), as well as under CA130460 awarded by the Department of Defense (DOD). The government has certain rights in the invention.

### BACKGROUND OF THE INVENTION

Tumor necrosis factor-related apoptosis inducing ligand (TRAIL) selectively induces death receptor (DR)-mediated apoptosis in cancer cells while sparing normal tissue, and therefore has garnered great interest as a possible cancer therapy. Ligands and agonists of DRs, such as recombinant human (rh) TRAIL, engineered TRAIL analogs, TRAIL fusion proteins, agonistic DR antibodies, and agonistic small molecules or peptidic molecules binding DRs have all gained interest as possible cancer therapies.

Various cancers are TRAIL-resistant. To address tumor heterogeneity, the use of TRAIL sensitizers has been contemplated as a way to overcome TRAIL resistance and effectively treat TRAIL-resistant primary tumors. Conventional cytotoxic agents have been shown to sensitize TRAIL resistant tumors; however, such agents are both toxic and have failed to show synergy when combined with TRAIL-based agents in clinical studies. Moreover, tumors must be continuously sensitized to maximize TRAIL-induced apoptosis, but frequent systemic injections of such toxic agents are not practical in the clinic. Therefore, there is a need in the field to effectively sensitize TRAIL-resistant tumors, while avoiding toxicity and numerous injections.

### SUMMARY OF THE INVENTION

The present invention is based, at least in part, upon discovery of an effective combinatorial administration of select kinase inhibitors (KIs), particularly oral KIs, and long-acting death receptor agonists (as described and exemplified herein), like recombinant PEGylated trimeric isoleucine-zipper fused TRAIL (TRAIL_{PEG}), for treatment of cancers, particularly for treatment of cancers that are or are at risk of developing TRAIL resistance. In addition, certain aspects of the invention describe a method of screening for the combinatorial effect of KIs with TRAIL-based agonists.

In one aspect, the invention provides a method for sensitizing a cancer of a subject to treatment with a death receptor agonist, the method involving administering a kinase inhibitor (KI) to the subject in an amount sufficient to sensitize the cancer to treatment with a long-acting death receptor agonist, thereby sensitizing the cancer of the subject to treatment with a death receptor agonist.

In one embodiment, the KI is A-674563, Afatinib (BIBW2992), Apatinib, AST-1306, AT7519, AT9283, AZ 960, AZD3463, AZD5438, BGJ398, BMS-265246, Bosutinib, Canertinib, CCT137690, CHIR-124, CHIR-98014, CP-673451, CYT387, Dacomitinib, Dactolisib, Dasatinib, Dinaciclib, Dovitinib, ENMD-2076, Flavopiridol HCL, Foretinib, GSK1904529A, Idelalisib, INCB28060, Lapatinib, Lenvatinib, Linifanib, Linsitinib, LY2784544, MGCD-265, Milciclib, Neratinib, OSI-930, Pazopanib, PD168393, PD98059, Pelitinib, PF-00562271, PHA-767491, PHA-793887, PIK-75, Regorafenib, Seliciclib, Saracatinib, SGX-523, SNS-032, Sunitinib Malate, TAK-901, TG101209, Tyrphostin, U0126-EtOH, Volasertib, WZ4002 or ZM 306416, or a combination thereof.

Optionally, the KI target is VEGFR, Src, MEK, PI3K, EGFR, CDK, JAK, CDK or c-Met, or a combination thereof.

In one embodiment, the KI is orally administered, optionally at a dosage of 1 mg to 1 g per tablet.

In another embodiment the cancer is sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer including prostate adenocarcinoma, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, retinoblastoma, multiple myeloma, rectal carcinoma, thyroid cancer, head and neck cancer, brain cancer, cancer of the peripheral nervous system, cancer of the central nervous system, neuroblastoma, colorectal adenocarcinoma or cancer of the endometrium, or a combination thereof.

In an additional embodiment, the cancer is a TRAIL-resistant cancer.

In one embodiment, the method further involves administering a long-acting death receptor agonist to the subject.

Optionally, the death receptor agonist is systemically (e.g., intravenously or subcutaneously) administered.

In certain embodiments, the death receptor agonist and the KI are co-administered.

In one embodiment, the death receptor agonist includes a tumor necrosis factor (TNF)-related apoptosis-inducing ligand (TRAIL), a TRAIL analogue, death receptor agonist antibodies, or a derivative thereof.

In another embodiment, the death receptor agonist includes human recombinant TRAIL, a human TRAIL analogue, or a derivative thereof

Optionally, the death receptor agonist includes native TRAIL, a native TRAIL analogue, or a derivative thereof.

In certain embodiments, the death receptor agonist is selectively attached to a polymer.

In one embodiment, the polymer includes polyethylene glycol (PEG), or derivative thereof. In a related embodiment, the PEG is methoxypolyethylene glcycol succinimidyl propionate, methoxypolyethylene glycol succinate N-hydroxysuccinimide, methoxypolyethylene glycol propionaldehyde, methoxypolyethylene glycol maleimide, or multiple-branched polyethylene glycol.

In certain embodiments, the death receptor agonist includes PEGylated trimeric isoleucine-zipper fused TRAIL (TRAILp_{EG}).

Optionally, the cancer is colorectal cancer.

In certain embodiments, the KI is OSI-930, Pazopanib, Saracatinib (AZD0530), Bosutinib (SKI-606), Dasatinib, Regorafenib (BAY 73-4506), ENMD-2076, PD98059, U0126-EtOH, CAL-101 (Idelalisib, GS-1101), BEZ235 (NVP-BEZ235, Dactolisib), or a combination thereof and the cancer is colorectal adenocarcinoma.

In other embodiments, the KI is Pelitinib (EKB-569), AT9283, Dasatinib, Canertinib (CI-1033), PHA-793887, Roscovitine (Seliciclib,CYC202), SNS-032 (BMS-387032), PIK-75, LY2784544, PF-00562271, AZ 960, CYT387, Volasertib (BI 6727), A-674563, Flavopiridol HCl, TG101209, TAK-901, BMS-265246, CHIR-124, Dacomitinib (PF299804, PF299), PHA-767491, CCT137690, CHIR-98014, Milciclib (PHA-848125), Dinaciclib (SCH727965), Dovitinib (TKI-258) Dilactic Acid, or a combination thereof and the cancer is breast cancer.

In additional embodiments, the KI is WZ4002, AT7519, SNS-032 (BMS-387032), GSK1904529A, Linifanib (ABT-869), Afatinib (BIBW2992), Lapatinib (GW-572016) Ditosylate, Apatinib, AZD5438, Flavopiridol HCl, CP-673451, BMS-265246, BGJ398 (NVP-BGJ398), CHIR-124, Dinaciclib (SCH727965), or a combination thereof and the cancer is lung cancer.

In further embodiments, the KI is Afatinib (BIBW2992), AST-1306, AZD3463, CP-673451, Dacomitinib (PF299804, PF299), Foretinib (GSK1363089), INCB28060, Lapatinib (GW-572016) Ditosylate, Lenvatinib (E7080), MGCD-265, Neratinib (HKI-272), OSI-906 (Linsitinib), PD168393, Regorafenib (BAY 73-4506), SGX-523, Sunitinib Malate, Tyrphostin 9, Tyrphostin AG 1296, Tyrphostin AG 879, WZ4002, ZM 306416, or a combination thereof and the cancer is prostate adenocarcinoma.

Another aspect of the invention provides a method for sensitizing a cancer cell to respond to a death receptor agonist, the method involving contacting the cancer cell with a kinase inhibitor (KI) in an amount sufficient to sensitize the cancer cell to respond to a death receptor agonist, thereby sensitizing the cancer cell to respond to a death receptor agonist.

In one embodiment, the cancer cell is contacted in vitro.

An additional aspect of the invention provides a method for identifying a kinase inhibitor (KI) capable of sensitizing a cancer cell to a death receptor agonist involving contacting the cancer cell with a KI; contacting the cancer cell with a death receptor agonist; and detecting cell death or a marker of apoptosis in the cancer cell administered the KI, as compared to an appropriate control cell, thereby identifying a kinase inhibitor (KI) capable of sensitizing a cancer cell to a death receptor agonist.

In one embodiment, the cancer cell is contacted with the KI for at least 3 hours, optionally for 6 hours or more, 12 hours or more, or 24 hours or more, in advance of contacting the cancer cell with the death receptor agonist.

Optionally, cell death or a marker of apoptosis in the cancer cell is measured by a cell death assay, an imaging agent, or by Western blot.

A further aspect of the invention provides a method for treating or preventing a cancer in a subject, the method involving administering a kinase inhibitor and a death receptor agonist to the subject in an amount sufficient to treat or prevent the cancer in the subject, thereby treating or preventing the cancer in the subject.

In one embodiment, the KI and the death receptor agonist act synergistically to treat or prevent the cancer in the subject.

### Definitions

By “agent” is meant any small compound, antibody, nucleic acid molecule, or polypeptide, or fragments thereof.

An “agonist” as used herein is a molecule which enhances the biological function of a protein. The agonist may thereby bind to the target protein to elicit its functions. However, agonists which do not bind the protein are also envisioned. The agonist may enhance the biological function of the protein directly or indirectly. Agonists which increase expression of certain genes are envisioned within the scope of particular embodiments of the invention. Suitable agonists will be evident to those of skill in the art. For the present invention, it is not necessary that the agonist enhances the function of the target protein directly. Rather, agonists are also envisioned which stabilize or enhance the function of one or more proteins upstream in a pathway that eventually leads to activation of targeted protein. Alternatively, the agonist may inhibit the function of a negative transcriptional regulator of the target protein, wherein the transcriptional regulator acts upstream in a pathway that eventually represses transcription of the target protein.

By “ameliorate” is meant decrease, suppress, attenuate, diminish, arrest, or stabilize the development or progression of a disease or disorder.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Death receptors” form a subclass of the Tumor Necrosis Factor Receptor (TNFR) superfamily, which encompasses eight members: Fas, TNFR1, neurotrophin receptor (p75NTR), ectodysplasin-A receptor (EDAR), death receptor (DR) 3, DR4, DRS, and DR6. Most of the death receptors have their corresponding natural ligands identified: TNFR1 can be activated by TNF, Fas is activated by Fas ligand (FasL), p75NTR is activated by nerve growth factor (NGF, gene ID: 4803). One ligand for EDAR is ectodysplasin-A (EDA, gene ID: 1896). DR3 can be activated by Apo3L (TWEAK/TNFSF12, gene ID: 8742), TL1A/VEGI (vascular endothelial growth inhibitor/TNFSF15, gene ID: 9966), while DR4 and DRS share the same ligand, TNF-related apoptosis-inducing ligand (TRAIL). The ligand for DR6 has not been identified. These ligands, their variants or any molecule that mimic the effect of the natural ligand is considered as a death receptor agonist. Each of these natural ligands and agonists thereof is considered a death receptor agonist.

A “death receptor agonist” is defined herein as any molecule which is capable of inducing pro-apoptotic signaling through one or more of the death receptors. The death receptor agonist may be selected from the group consisting of antibodies, death ligands, cytokines, death receptor agonist expressing vectors, peptides, small molecule agonists, cells (for example stem cells) expressing the death receptor agonist, and drugs inducing the expression of death ligands.

Exemplary death receptor agonists are capable of binding to a death receptor and inducing apoptosis or programmed cell death through one or more intracellular pathways. Exemplary well studied death receptor agonists include members of the TNF ligand family, which can play key roles in regulatory and deleterious effects on immune tolerance, in addition to both protective and pathogenic effects on tissues (Rieux-Laucat et al., 2003, Current Opinion in Immunology 15:325; Mackay and Ambrose, 2003, Cytokine and growth factor reviews, 14: 311; Mackay and Railed, 2002, Current Opinion in Immunology, 14: 783-790). Examples of such proteins include Tumor necrosis factor-related apoptosis inducing ligand (TRAIL), Fas ligand (FasL) and Tumor Necrosis Factor (TNF). Exemplary death receptor agonists induce apoptosis upon binding to transmembrane, death domain containing receptors. For example, TRAIL binds to death receptor 4 (DR4; TRAIL receptor 1) and 5 (DRS; TRAIL receptor 2). Three other TRAIL-binding receptors exist, but are considered to be “decoy receptors” as they appear to be unable to transmit an apoptotic signal. Decoy receptor 1 (DcR1) appears to lack the transmembrane and intracellular domains and is anchored to the plasma membrane via a glycosylphosphatidylinositol-tail. Decoy receptor 2 (DcR2) possesses a truncated and apparently non-functional death domain, while the third decoy receptor, osteoprotegerin is a secreted, soluble receptor. Fas ligand induces apoptosis by binding to Fas (also known as CD95 or Apo-1), while DcR3 sequesters FasL from Fas. Another death receptor agonist, TNF can induce apoptosis by binding to TNF-receptor I (also known as TNFRI or TNFR55).

“Detect” refers to identifying the presence, absence or amount of the analyte to be detected.

By “effective amount” is meant the amount of an agent required to ameliorate the symptoms of a disease relative to an untreated patient. The effective amount of active agent(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “marker” is meant any protein or polynucleotide having an alteration in expression level or activity that is associated with a disease or disorder.

By “modulate” is meant alter (increase or decrease). Such alterations are detected by standard art known methods such as those described herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduces” is meant a negative alteration of at least 10%, 25%, 50%, 75%, or 100%.

As used herein, “obtaining” as in “obtaining an agent” includes synthesizing, purchasing, or otherwise acquiring the agent.

By “subject” is meant a mammal, including, but not limited to, a human or non-human mammal, such as a bovine, equine, canine, ovine, or feline.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control, e.g., a standard or control condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof

A “therapeutically effective amount” is an amount sufficient to effect beneficial or desired results, including clinical results. An effective amount can be administered in one or more administrations.

The term “theranostics” refers to efforts in clinics to develop more specific, individualized therapies for various diseases, and to combine diagnostic and therapeutic capabilities into a single agent and/or unified process/regimen.

The term “TRAIL” also includes TRAIL heterodimers, homodimers, heteromultimers, or homomultiniers of any one or more TRAIL or any other polypeptide, protein, carbohydrate, polymer, small molecule, linker, ligand, or other biologically active molecule of any type, linked by chemical means or expressed as a fusion protein, as well as polypeptide analogues containing, for example, specific deletions or other modifications yet maintain biological activity.

The terms “tumor,” “solid tumor,” “primary tumor,” and “secondary tumor” refer to carcinomas, sarcomas, adenomas, and cancers of neuronal origin and, in fact, to any type of cancer which does not originate from the hematopoietic cells and in particular concerns: carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, retinoblastoma, multiple myeloma, rectal carcinoma, thyroid cancer, head and neck cancer, brain cancer, cancer of the peripheral nervous system, cancer of the central nervous system, neuroblastoma, cancer of the endometrium, as well as metastasis of all the above.

A “Tumor Necrosis Factor family member” or a “Tumor Necrosis Factor ligand family member” is any cytokine which is capable of activating a Tumor Necrosis Factor receptor. “TRAIL protein”, as used herein, encompasses both the wild-type TRAIL protein and TRAIL variants.

By “variant” death receptor agonist, it is meant that the death receptor agonist differs in at least one amino acid position from the wild type sequence of the death receptor agonist. By “variant” TRAIL protein it is meant that the TRAIL protein differs in at least one amino acid position from the wild type TRAIL protein (also known as TNFSF1O, TL2; APO2L; CD253; Apo-2L), Entrez GenelD: 8743; accession number NM 003810.2; UniProtKB/Swiss-Prot: P50591; UniProtKB/TrEMBL: Q6IBA9.

Any compositions or methods provided herein can be combined with one or more of any of the other compositions and methods provided herein.

Other features and advantages of the invention will be apparent to those skilled in the art from the following detailed description and claims.

### BRIEF DESCRIPTION OF THE DRAWINGS

FIG. 1 depicts a schematic diagram of novel TRAIL-based therapy that combines long-acting TRAIL_{PEG} and orally active TRAIL sensitizer.

FIG. 2A depicts a bar graph showing that when primary cancer cells are treated with TRAIL they demonstrate resistance to TRAIL-induced apoptosis. Quantified cell death data after TRAIL (1 μg/mL) treatment for 24 hours in various cancer cell types are shown. Human tumor cell lines include: colon (HT-29, SW620, HCT116), prostate (PC3), breast (MDA-MD-231, MCF-7), lung (A549). HCT116 represents a TRAIL-sensitive colorectal tumor for comparison. HEK293T is a normal human kidney cell line.

FIG. 2B depicts a bar graph showing quantified cell death after analyzing synergized cell death induced by TRAIL_{PEG} (1 μg/mL for 3 hr) and kinase inhibitors (KIs) in HT29 (human colon tumor cell line) and PC3 (human prostate tumor cell line) cells individually pretreated with selected 355 KIs. Relative cell death rates were calculated by [KI+TRAIL_{PEG}]/[KI alone] after separate MTT assays. The arrows indicate KIs with >60% cell death; n=3.

FIG. 3A depicts a bar graph showing that regorafenib (an oral, multi-kinase inhibitor) enhanced TRAIL_{PEG} -induced apoptosis in colorectal cancer (CRC) cells. Quantified cell death after combinatorial treatment with regorafenib and TRAIL_{PEG} (1 μg/mL) in HT-29 cells is shown.

FIG. 3B depicts a Western blot analysis of HT29 cells with regorafenib alone or in combination with TRAIL_{PEG}.

FIG. 4A depicts a bar graph showing qPCR analysis of death receptors (DRs) and decoy receptors (DcRs) in HT29 cells treated with regorafenib for 24 hours or 48 hours as indicated; *P<0.05, **P<0.01, ***P<0.001 versus control.

FIG. 4B depicts a Western blot analysis showing up-regulation of DR4 at 48 hours post-regorafenib treatment in HT29 cells, while the anti-apoptotic BCL-2 family members MCL-1 and BCL-2 were down-regulated.

FIG. 5 depicts a bar graph showing results of tumor volumes in mice bearing TRAIL-resistant HT29 tumors treated with three rounds of TRAIL_{PEG} (150 μg, i.v), oral regorafenib (10 mg/kg) or oral regorafenib with TRAIL_{PEG} within days (12-17) after tumor inoculation. Mice were sacrificed on day 27. The regorafenib/TRAIL_{PEG} combination significantly suppressed tumor growth compared to the individual treatments (n=5). *P<0.05, **P<0.01. FIG. 6A depicts a Western blot analysis of LNCAP prostate cancer cells with regorafenib alone or in combination with TRAIL_{PEG}.

FIG. 6B depicts a Western blot analysis of DU145 prostate cancer cells with regorafenib alone or in combination with TRAIL_{PEG}.

FIG. 6C depicts a Western blot analysis of PC-3 prostate cancer cells with regorafenib alone or in combination with TRAIL_{PEG}.

FIG. 7 is a bar graph showing that regorafenib enhanced TRAILPEG-induced apoptosis in prostate cancer cells. Quantified cell death after combinatorial treatment with regorafenib (5 μM) and TRAIL_{PEG} (1 μg/mL) in various prostate cancer cells is shown. *P<0.05, **P<0.01, ***P<0.001 versus control (regorafenib only).

### DETAILED DESCRIPTION OF THE INVENTION

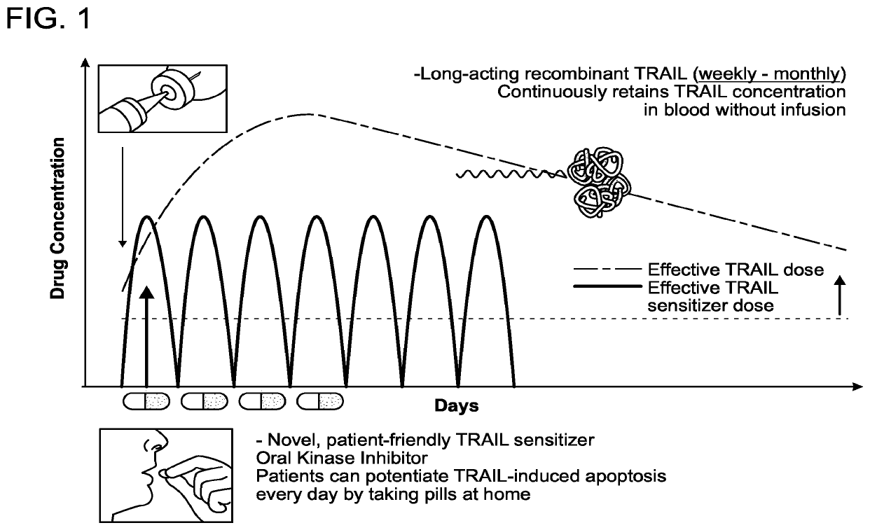

The invention is based, at least in part, upon the discovery of molecularly-targeted, reduced toxicity kinase inhibitors (Ms; where toxicity is reduced as compared to, e.g., traditional cytotoxic agents, i.e., chemotherapeutics) as a TRAIL-sensitizing strategy to treat cancer patients. In particular, novel TRAIL-based regimens that include combinatorial therapy with kinase inhibitor(s) were identified and continue to be a focus (FIG. 1). In exemplary such combination therapies, cancer patients can be treated infrequently with long-acting DR agonists, while conveniently sensitizing cancers to TRAIL therapy using kinase inhibitors, that, optionally, can even be administered orally (e.g., via daily oral pills). Such reduced toxicity and patient-friendly approaches were newly identified as highly beneficial to cancer patients, and possess the potential to replace/displace current therapies that require burdensome and frequent injections of toxic chemotherapeutics, which often can only be administered at a clinic.

The invention also describes a method of screening the combinatorial efficacy of Ms and TRAIL-based agents. After screening over 350 safety-confirmed (e.g., low toxicity) Ms with TRAIL_{PEG} treatment in human colon, prostate, lung and breast cancer cells, approximately 1% of the KIs screened were identified as having induced strong DR-mediated apoptosis via unknown mechanisms, and superior TRAIL sensitization in TRAIL-resistant cancer cells. In particular, a few FDA-approved KIs were discovered as potent novel TRAIL sensitizers, even though their precise roles in TRAIL sensitization have yet to be defined. It is therefore contemplated to use select KIs and kinases as sensitizers of TRAIL in different types of cancer cells, and significant steps have been made herein towards defining a universal TRAIL-based therapeutic approach for cancer therapy.

Combined with the high unmet clinical need for less toxic anticancer therapies, the discoveries of the instant invention warrant clinical translation as both a unique long-acting, less toxic TRAIL combination therapy. Development of diverse TRAIL therapies for a wide range of cancers, including lung, breast, prostate and rare cancers, is contemplated.

Potencies of KI and long-acting TRAIL-based agent combinations are validated in different types of xenograft models towards development of an anticancer biologic with significantly reduced side effects and improved patient compliance. It is contemplated herein that a long-acting TRAIL-based formulation can be transferred to the next step of clinical translation for extensive pharmacokinetic and pharmacodynamic studies at different dosing regimens, multiple doses in diverse animal models, mass production and toxicity studies. The instant invention also demonstrates a screening method for KIs for TRAIL-based therapy and allow for development of a thorough understanding of select KI and individual kinase roles upon TRAIL signaling and DR-mediated apoptosis ate a molecular level, both in cells and in vivo.

Additional features of the invention are set forth below and elsewhere herein.

TRAIL Tumor necrosis factor (TNF)-related apoptosis-inducing ligand (TRAIL) is a member of the TNF family, and is a transmembrane protein that participates in apoptosis. TRAIL is a protein consisting of 281 amino acids in which an extracellular domain comprising amino acids from arginine at position 114 to glycine at position 281 affects apoptosis. Three molecules of TRAIL form a structurally modified trimer. The TRAIL trimer assembles with receptors participating in cell death to induce apoptosis. A major difference between TRAIL and other members of the TNF superfamily is its ability not to induce cell death at normal tissues. Since TNF affects normal cells and also induces the death of cancer cells and over-activated immune cells, it has limited applicability. In contrast, TRAIL induces apoptosis in a wide range of cancer cells and over-activated immune cells with little effect on normal cells. This is due to the differential expression of TRAIL receptors between cell types.

Without wishing to be bound by theory, TRAIL induces apoptosis by interacting with its receptors. Currently, four human receptors for TRAIL have been identified, including death receptor 4 (DR4), death receptor 5 (DRS), decoy receptor 1 (DcR1), decoy receptor 2 (DcR2), and osteoprotegrin (OPG). TRAIL induces death via caspase-dependent apoptosis upon binding to DR4 and DRS, which both contain a conserved death domain (DD) motif. DcR1 and DcR2 act as decoys for their ability to inhibit TRAIL-induced apoptosis when overexpressed. DcR1 and DcR2 have close homology to the extracellular domains of DR4 and DRS. DcR2 has a truncated, nonfunctional cytoplasmic DD, while DcR1 lacks a cytosolic region and is anchored to the plasma membrane through a glycophospholipid moiety. The cytoplasmic domain of DcR2 is functional and activates NF-κB which leadings to transcription of genes known to antagonize the death signaling pathway and/or to promote inflammation. Ligand binding to DR4 triggers receptor trimerization and clustering of its intracellular death domains, resulting in the formation of a death inducing complex (DISC).

The DISC recruits adaptor molecules and initiates the binding and activation of caspases to induce apoptosis. Inducing or restoring signaling through TRAIL receptors is an anticancer strategy; TRAIL has also been shown to inhibit auto antigen-specific T cells indicating that it may suppress autoimmune responses.

In addition to toxicity toward some normal cells, TRAIL has a short half-life in vivo, and has different half-lives according to the species of animals used in tests. For example, TRAIL has been reported to have a half-life of several minutes in rodents and about 30 minutes in apes (H. Xiang, et al. Drug Metabolism and Disposition 2004, 32, 1230-1238). In particular, most of TRAIL is rapidly excreted via the kidneys.

### TRAIL Therapy in the Clinic

One highly attractive feature of TRAIL is its safety (Ashkenazi A, et al., J Clin Invest. 1999; 104(2):155-162, Yee L, et al., J Clin Oncol. 2007; 25(18s), and Lemke J, et al., Cell Death Differ. 2014; 21(9):1350-1364), and its inherent cancer-selectivity. TRAIL selectively induces apoptosis by binding to its DRs, TRAIL-R1/DR4 and TRAIL-R2/DR5, which are widely expressed in most cancers while sparing normal tissues (Ashkenazi A. Nat Rev Cancer. 2002; 2(6):420-430, de Vries E G, et al., Clin Cancer Res. 2006; 12(8):2390-2393, and Ashkenazi A, et al., J Clin Invest. 2008; 118(6):1979-1990). Recently-initiated clinical studies of dulanermin (recombinant TRAIL) in cancer patients revealed broad tolerability but unfortunately failed to demonstrate a robust therapeutic benefit (Lemke J, et al., Cell Death Differ. 2014; 21(9):1350-1364, and Soria J C, et al., J Clin Oncol. 2011; 29(33):4442-4451). Without wishing to be bound by theory, several factors that are likely to account for this unexpected clinical outcome have since been discussed: (1) dulanermin is a relatively weak DR agonist with a short half-life (e.g., 5 min in rodents; Kelley S K, et al., J Pharmacol Exp Ther. 2001; 299(1):31-38, and Ashkenazi A, et al., J Clin Oncol. 2008; 26(21):3621-3630), (2) most primary cancers are resistant to TRAIL monotherapy, (Newsom-Davis T, et al., Apoptosis. 2009; 14(4):607-623, and Dimberg L Y, et al., Oncogene. 2013; 32(11):1341-1350) and (3) diagnostic approaches are lacking to identify patients who will benefit from TRAIL treatment. The majority of primary cancer cells are TRAIL-resistant. Mechanisms of TRAIL resistance are distinct among cancer cell types; however, they commonly comprise of reduced cell surface DR expression, inhibited caspase-8 activation, up-regulated anti-apoptotic molecules such as Bcl-2 and the inhibitors of apoptosis (IAP) family proteins, and reduced expression of pro-apoptotic markers like Bax/Bak (Hellwig CT, et al., Mol Cancer Ther. 2012; 11(1):3-13, and Voelkel-Johnson C. Nat Rev Urol. 2011; 8(8):417-427).

Exploring TRAIL sensitizers has continued for the past fifteen years; however, none of the reported TRAIL combinations have exhibited proven efficacy in humans. The role of diverse molecules like anticancer agents in sensitizing TRAIL-resistant cancer cells have been investigated and introduced as an addition to TRAIL monotherapy. Certain TRAIL-based combinations were well validated in vitro and in a few in vivo cancer models; however, they failed to demonstrate a similar synergy in cancer patients (Lemke J, et al., Cell Death Differ. 2014; 21(9):1350-1364). Integrating recent findings from basic and clinical studies related to TRAIL biology and therapy, a TRAIL-based therapeutic approach that overcomes the short half-life and TRAIL-resistance seen in therapies so far is viewed as a significant and unexpected discovery, and is described herein.

### Introducing a Potent and Patient-Friendly TRAIL Therapy

Significant advantages of utilizing recombinant TRAIL, as contrasted with TRAIL agonistic antibodies, are: TRAIL can simultaneously target both DR4/DRS, and TRAIL is found in the body so there are limited concerns about its safety or immunogenicity (Lawrence D, et al., Nat Med. 2001; 7(4):383-385). Recent clinical studies of TAS266, a tetravalent nanobody targeting DRS, were terminated early because of hepatotoxicity of antibodies in patients (Papadopoulos K P, et al., Cancer Chemother Pharmacol. 2015). To overcome the short half-life and low potency of dulanermin, focus has been upon (1) engineering TRAIL to develop a highly stable, potent, yet safe TRAIL, (Chae S Y, et al., Mol Cancer Ther. 2010; 9(6):1719-1729, Kim T H, et al., J Control Release. 2011; 150(1):63-69, Lim S M, et al., Biomaterials. 2011; 32(13):3538-3546, Kim T H, et al., Bioconjug Chem. 2011; 22(8):1631-1637, and Kim T H, et al., Angew Chem Int Ed Engl. 2013; 52(27):6880-6884), (2) investigating new TRAIL sensitizers with less systemic toxicity (Jiang H H, et al., Biomaterials. 2011; 32(33):8529-8537), and (3) exploring TRAIL signaling.

Soluble trimeric isoleucine-zipper fusion TRAIL (iLZ-TRAIL) is a potent variant of TRAIL, as compared to a native-type TRAIL like dulanermin. A long-acting PEGylated iLZ-TRAIL (TRAIL_{PEG}) was developed and was demonstrated to possess extended half-life in non-human primates and safety in primary human hepatocytes (Oh Y et al., Hepatology. 2015 Dec. 28. doi: 10.1002/hep.28432). Continuous sensitization of TRAIL-resistant cancer cells in patients is now contemplated as a logical way to maximize TRAIL-based therapy. Diverse cytotoxic agents have been shown to sensitize cancer cells to TRAIL. However, for clinical application, frequent injections of such toxic agents are not possible. In addition, clinical studies of short-acting dulanermin combined with chemotherapy did not reveal improved anticancer activity in lung and colon cancer patients (Soria J C, et al., J Clin Oncol. 2011; 29(33):4442-4451).

### TRAIL-Conjugates

As identified herein, combinatorial administration of select kinase inhibitors (KIs), particularly oral KIs, with ligands and agonists of agonistic TRAIL receptors, particularly long-acting TRAIL receptor agonists, like recombinant PEGylated trimeric isoleucine-zipper fused TRAIL (TRAIL_{PEG}), were effective for treatment of cancers, particularly for treatment of cancers that are or are at risk of developing TRAIL resistance, when such combinatorial agents/formulations were administered as a single dose with a regularity of daily or less frequently—e.g., daily, every other day, twice weekly, optionally once weekly, once every two weeks, once monthly or even less than once monthly.

In certain embodiments, the ligand or agonist does not require a delivery vehicle such as a controlled or sustained release formulation to be effective.

The ligands and agonists disclosed herein are typically TRAIL conjugates that include a TRAIL peptide, or mimic, optionally TRAIL or a fragment, variant, or fusion thereof, linked to a conjugate molecule that extends the in vivo half-life of the TRAIL-conjugate when compared to the TRAIL fragment, variant, or fusion in the absence of the conjugate molecule.

TRAIL Peptides and Analogues

TRAIL-conjugates include a TRAIL domain, which is typically a TRAIL peptide, analogue, or mimic, optionally TRAIL or a fragment, variant, or fusion thereof to which a conjugate molecule is linked.

TRAIL

TRAIL/Apo2L (TNFSF10) was originally identified in searches of EST databases for genes with homology to known TNF superfamily ligands (Benedict et al., J. Exp. Med., 209(11):1903-1906 (2012)). In humans, TRAIL binds two proapoptotic death receptors (DRs), TRAIL-R1 and -R2 (TNFRSF10A and 10B), as well as two other membrane receptors that do not induce death and instead may act as decoys for death signaling. TRAIL binding to its cognate DRs induces formation of a death-inducing signaling complex, ultimately leading to caspase activation and initiation of apoptosis (Benedict et al., J. Exp. Med., 209(11):1903-1906 (2012)).

In some embodiments, the TRAIL conjugate includes a TRAIL peptide, or an agonistic TRAIL receptor binding fragment or variant thereof.

Nucleic acid and amino acid sequence for human TRAIL are known in the art. For example, an amino acid sequence for human TRAIL is MAMMEVQGGPSLGQTCVLIVIFTVLLQSLCVAVTYVYFTNELKQMQDKYSKSGIAC FLKEDDSYWDPNDEESMNSPCWQVKWQLRQLVRKMILRTSEETISTVQEKQQNISPL VRERGPQRVAAHITGTRGRSNTLSSPNSKNEKALGRKINSWESSRSGHSFLSNLHLRN GELVIHEKGFYYIYSQTYFRFQEEIKENTKNDKQMVQYIYKYTSYPDPILLMKSARNS CWSKDAEYGLY SIYQGGIFELKENDRIFVSVTNEHLIDMDHEASFFGAFLVG (SEQ ID NO:1; UniProtKB database accession no. P50591 (TNF10 HUMAN)). In some embodiments, the TRAIL conjugate includes a TRAIL peptide including or having the amino acid sequence of SEQ ID NO:1.

Optionally, the TRAIL is a soluble TRAIL. Endogenous, full-length TRAIL includes a cytoplasmic domain, a transmembrane domain, and an extracellular domain. Typically, soluble TRAIL is a fragment of full-length TRAIL without the cytoplasmic domain and the transmembrane domain. Therefore, soluble TRAIL can be the extracellular domain of TRAIL (e.g., extracellular domain of SEQ ID NO:1), or a functional fragment thereof. A consensus extracellular domain for the TRAIL of SEQ ID NO:1 is amino acids 39-281 of SEQ ID NO:1. Therefore, in some embodiments, the TRAIL conjugate includes a TRAIL peptide including or having amino acids 39-281 of SEQ ID NO:1, or a functional fragment or variant thereof.

In some embodiments, the TRAIL conjugate includes a functional fragment or variant of SEQ ID NO:1 that act as an agonist signaling through TRAIL-R1 and/or TRAIL-R2. The fragment or variant of SEQ ID NO:1 can have 50, 60, 70, 75, 80, 85, 90, 95, 96, 97, 98, 99, or more than 99% sequence identity to SEQ ID NO:1.

Optionally, the functional fragment or variant thereof includes the extracellular domain of SEQ ID NO:1, or a functional fragment thereof. It is believed that the C-terminal 150 amino acid of TRAIL includes the receptor binding domain. Therefore, in some embodiments, the functional fragment includes amino acids 132-281 of SEQ ID NO:1. In other particular embodiments, the fragment is amino acids 95-281, or amino acids 114-281 of SEQ ID NO:1.

Variants can have one or more substitutions, deletions, or additions, or any combination thereof relative to SEQ ID NO:1. In some embodiments, the variant is a naturally occurring alternative sequence, splice variant, or substitution, addition or deletion variant, or the extracellular domain is a functional fragment of an alternative sequence, splice variant, or substitution, addition or deletion variant. Naturally occurring alternative sequences and variants are disclosed in UniProtKB database accession no. P50591 (TNF10 HUMAN), version 140 (last modified Jan. 22, 2014.

The Trail proteins described herein can be made using standard techniques for isolation of natural or recombinant proteins, and chemically modified as described herein.

TRAIL Analogues

TRAIL can interact with its receptors as a trimer. Therefore, in some embodiments, the ligand or agonist used in the methods disclosed herein is, or can form, a multimer, optionally a trimer. The trimer can be a homotrimer, or a heterotrimer.

The TRAIL conjugate can include a TRAIL analogue, or an agonistic TRAIL receptor binding fragment or variant thereof. TRAIL analogues are known in the art. In certain embodiments, the analogues have increased affinity or specificity for one or more agonistic TRAIL receptors (e.g., TRAIL-R1 (DR4) and/or TRAIL-R2 (DRS)), reduced affinity or specificity for one or more antagonistic or decoy TRAIL receptors (e.g., receptors DcR1 and DcR2) or a combination thereof compared to wildtype or endogenous TRAIL.

In some embodiments, the analogue is a DR4-selective mutant of wildtype TRAIL. DR-4 selective mutants are known in the art and disclosed in, for example, Tur, The Journal of Biological Chemistry, 283(29):20560-8 (2008). In a particular embodiments, the analogue is a variant of SEQ ID NO:1 having a D218H or a D218Y substitution, or a functional fragment thereof (e.g., the extracellular domain).

In some embodiments, the analogue is a DRS-selective mutant of wildtype TRAIL. Particular DR-5-selective mutants include variants of SEQ ID NO:1 having D269H, D269H/E195R, or D269H/T214R, and functional fragments thereof (e.g., the extracellular domain). Such variants are described in van der Sloot, Proceedings of the National Academy of Sciences of the United States of America, 103(23):8634-9 (2006).

TRAIL Fusion Proteins

The TRAIL conjugate can be a TRAIL fusion protein. TRAIL fusion polypeptides have a first fusion partner including all or a part of a TRAIL protein extracellular domain fused (i) directly to a second polypeptide or, (ii) optionally, fused to a linker peptide sequence that is fused to the second polypeptide. The fusion proteins optionally contain a domain that functions to dimerize or multimerize two or more fusion proteins. The peptide/polypeptide linker domain can either be a separate domain, or alternatively can be contained within one of the other domains (TRAIL polypeptide or second polypeptide) of the fusion protein. Similarly, the domain that functions to dimerize or multimerize the fusion proteins can either be a separate domain, or alternatively can be contained within one of the other domains (TRAIL polypeptide, second polypeptide or peptide/polypeptide linker domain) of the fusion protein. In one embodiment, the dimerization/multimerization domain and the peptide/polypeptide linker domain are the same.

Fusion proteins disclosed herein can be of formula I:

N—R₁—R₂—R₃—C

wherein “N” represents the N-terminus of the fusion protein, “C” represents the C-terminus of the fusion protein, “R₁” is a TRAIL polypeptide, “R₂” is an optional peptide/polypeptide linker domain, and “R₃” is a second polypeptide. Alternatively, R₃ may be the TRAIL polypeptide and R₁ may be the second polypeptide.

The fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

The presence of the second polypeptide can alter the solubility, stability, affinity and/or valency of the TRAIL fusion polypeptide. As used herein, “valency” refers to the number of binding sites available per molecule. In some embodiments, the second polypeptide contains one or more domains of an immunoglobulin heavy chain constant region, optionally having an amino acid sequence corresponding to the hinge, C_{H}2 and C_{H}3 regions of a human immunoglobulin Cγ1 chain or to the hinge, C_{H}2 and C_{H}3 regions of a murine immunoglobulin Cγ2a chain. In a particular dimeric fusion protein, the dimer results from the covalent bonding of Cys residue in the hinge region of two of the Ig heavy chains that are the same Cys residues that are disulfide linked in dimerized normal Ig heavy chains.

In a particular embodiment, the TRAIL fusion protein is a TRAIL-mimic including three TRAIL-protomer subsequences combined in one polypeptide chain, termed the single-chain TRAIL-receptor-binding domain (scTRAIL-RBD), as described in Gieffers, Molecular Cancer Therapeutics, 12(12):2735-47 (2013). Two of the so-called scTRAIL-RBDs, with three receptor binding sites each, can be brought in close proximity resulting in a multimeric fusion protein with a hexavalent binding mode. In some embodiments, multimerization is achieved by fusing the Fc-part of a human immunoglobulin G1 (IgG1)-mutein C-terminally to the scTRAIL-RBD polypeptide, thereby creating six receptor binding sites per drug molecule.

Forcing dimerization of scFv-scTRAIL based on scFv linker modification for a targeted scTRAIL composed predominantly of dimers (Db-scTRAIL) exceed the activity of nontargeted scTRAIL approximately 100-fold for some target cell types (Siegemund). Increased activity of Db-scTRAIL was also demonstrated on target-negative cells, indicating that, in addition to targeting, oligomerization equivalent to an at least dimeric assembly of standard TRAIL per se enhances apoptosis signaling. Therefore, in certain embodiments, the TRAIL fusion proteins have a multimerization domain, such as a dimerization or trimerization domain, or a combination thereof that can lead to, for example, dimeric, trimeric, or hexameric molecule.

Another fusion protein that facilitates trimer formation includes a receptor binding fragment of TRAIL amino-terminally fused to a trimerizing leucine or isoleucine zipper domain.

TRAIL fusion proteins and results of using the fusion proteins in functional assays are also described in, Wahl, Hepatology, 57(2):625-36 (2013).

Conjugates and Complexes

Certain disclosed TRAIL-conjugates also include a second conjugate molecule that is linked to the TRAIL domain.

Polyalkylene Oxides Such as PEG

Studies show that the pharmacokinetic and pharmacodynamic profiles of TRAIL can be improved using PEGylation (Kim, et al., Bioconjugate Chem., 22 (8), pp 1631-1637 (2011)). Studies show that TRAIL analogues derivatized with PEG maintain anti-cancer activity, while also exhibiting higher metabolic stabilities in plasma, extended pharmacokinetic profiles, and greater circulating half-lives (Chae, et al., Molecular cancer therapeutics 9(6):1719-29 (2010); Kim, et al., Bioconjugate chemistry, 22(8):1631-7 (2011); Kim, et al., Journal of pharmaceutical sciences 100(2):482-91 (2011); Kim, et al., Journal of controlled release: official journal of the Controlled Release Society 150(1):63-9 (2011)).

Therefore, in some embodiments, the TRAIL domain is derivatized with one or more ethylene glycol (EG) units, more optionally 2 or more EG units (i.e., polyethylene glycol (PEG)), or a derivative thereof. Derivatives of PEG include, but are not limited to, methoxypolyethylene glycol succinimidyl propionate, methoxypolyethylene glycol N-hydroxysuccinimide, methoxypolyethylene glycol aldehyde, methoxypolyethylene glycol maleimide and multiple-branched polyethylene glycol.

### PEGylated TRAIL

### Polyethylene Glycol

Polyethylene glycol (PEG) is a polymer having a structure of HO—(—CH₂CH₂O—)n-H. Due to its high hydrophilicity, PEG enables an increase in the solubility of drug proteins when linked thereto. In addition, when suitably linked to a protein, PEG increases the molecular weight of the modified protein while maintaining major biological functions, such as enzyme activity and receptor binding; thereby reducing urinary excretion, protecting the protein from cells and antibodies recognizing exogenous antigens, and decreasing protein degradation by proteases. The molecular weight of PEG, capable of being linked to proteins, ranges from about 1,000 to 100,000. PEG having a molecular weight higher than 1,000 is known to have very low toxicity. PEG having a molecular weight between 1,000 and 6,000 is distributed widely throughout the entire body and is metabolized via the kidney. In particular, PEG having a molecular weight of 40,000 is distributed in the blood and organs, including the liver, and is metabolized in the liver. Exemplary PEGs of the current subject matter include but are not limited to: methoxypolyethylene glcycol succinimidyl propionate, methoxypolyethylene glycol succinate N-hydroxysuccinimide, methoxypolyethylene glycol propionaldehyde, methoxypolyethylene glycol maleimide, and multiple-branched polyethylene glycol.

The precise number of EG or derivative units depends on the desired activity, plasma stability, and pharmacokinetic profile. For example, Kim, et al. (supra) reported that 2, 5, 10, 20, and 30K-PEG-TRAIL resulted in greater circulating half-lives of 3.9, 5.3, 6.2, 12.3, and 17.7 h respectively in mice, versus 1.1 h for TRAIL. In some embodiments, the molecular weight of the PEG is between about 1 and 100 kDa, optionally between about 1 and 50 kDa.

For example, the PEG can have a molecular weight of “N” kDa, wherein N is any integer between 1 and 100. The PEG can have a molecular weight of “N” Da, wherein N is any integer between 1,000 and 1,000,000. In a particular embodiment, the molecular weight of the PEG is “N” Da, wherein “N” is between 1,000 and 60,000, or more optionally between 5,000 and 40,000.

The pro-apoptotic agent can be conjugated with linear or branched PEG. Some studies have shown that proteins derivatized with branched PEG have extended in vivo circulation half-lives compared to linear PEG-proteins, thought to be due partly to a greater hydrodynamic volume of branched PEG-proteins (Fee, et al., Biotechnol Bioeng., 98(4):725-3 (2007)).

Peptide ligands can be derivatized at the C-terminus, or optionally at the N-terminus, using methods that are known in the art.

The TRAIL-PEG conjugates may be depicted by the following formula:

X-L-(PEG)ₙ, wherein X represents a TRAIL protein, L represents a linker, PEG represents a branched poly(ethylene glycol) chain, and n is an integer selected from 2, 3, 4, 5, 6, 7 or 8. In certain embodiments, n is 2.

The polyalkylene oxide can be coupled to the protein via a linker. The linker may be a polyalkylene oxide, and optionally connects two polyalkylene oxide polymers to the protein.

In a particular embodiment, the TRAIL-conjugate is a PEG-conjugate that includes a TRAIL domain including a truncated form of human TRAIL, for example, from arginine-114 to glycine-281 of the full-length form (1-281) of human TRAIL, and PEG having a molecular weight between 1,000 and 100,000 Daltons, and optionally between 5,000 and 50,000 Daltons.

N-terminal modified PEG-TRAIL conjugates can be obtained by reacting an N-terminal amine of the TRAIL domain with an aldehyde group of the PEG in the presence of a reducing agent. PEG and TRAIL can be reacted at a molar ratio (PEG/TRAIL) of 2 to 10, or optionally 5 to 7.5.

In certain embodiments, the TRAIL-conjugate includes a zipper amino acid motif, for example, an isoleucine zipper motif, that allows for trimer formation between three TRAIL-conjugate monomers.

The PEG chains are optionally, but not necessarily, of equal molecular weight. Exemplary molecular weight ranges for each PEG chain is between about 10 kDa and 60 kDa, and optionally about 20 kDa and 40 kDa. PEG40 is a branched PEG moiety was synthesized and has a molecular weight of 40 kDa: 20+20 kDa (each PEG chain).

A trimeric PEG moiety can consist of a branched PEG chain attached to a linker arm. A visual description of the trimer PEG moiety is provided immediately below.

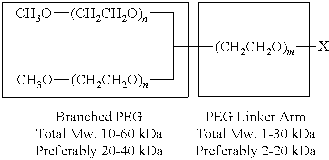

The following trimeric PEGs can be synthesized: YPEG42, YPEG43.5, YPEG45, YPEG50 and YPEG60.

YPEG42 is a trimeric PEG moiety which has a molecular weight of 42 kDa: (20+20 kDa) (branched PEG)+2 kDa (linker arm).

YPEG43.5 is a trimeric PEG moiety which has a molecular weight of 43.5 kDa: (20+20 kDa) (branched PEG)+3.5 kDa (linker arm).

YPEG45 is a trimeric PEG moiety which has a molecular weight of 45 kDa: (20+20 kDa) (branched PEG)+5 kDa (linker arm).

YPEG50 is a trimeric PEG moiety which has a molecular weight of 50 kDa: (20+20 kDa) (branched PEG)+10 kDa (linker arm).

YPEG60 is a trimeric PEG moiety which has a molecular weight of 60 kDa: (20+20 kDa) (branched PEG)+20 kDa (linker arm).

Linker Moiety

The protein or peptide is covalently joined to the branched PEG moiety via a linker. The linker is a polymer, and generally has an atomic length of at least 800 angstroms. Typically, the linker has an atomic length from about 800 to about 2,000 angstrom, from about 800 to about 1,500 angstrom, from about 800 to about 1,000 angstrom, or from about 900 to about 1,000 angstrom. It is to be appreciated that the atomic distances listed above refer to fully extended polymers, and that when in the solid state or solution the linker may fold or curl in ways such that the actual distance between the branched PEG and protein or peptide is less than the atomic lengths listed above.

In certain embodiments, the linker is a poly(ethylene glycol) derivative with a molecular weight between about 1 kDa to 30 kDa, optionally from about 2 kDa to 20 kDa. A linker may also be a natural or unnatural amino acid of at least 80 units in length.

PEG alternatives for the linker include synthetic or natural water-soluble biocompatible polymers such as polyethylene oxide, polyvinyl alcohol, polyacrylamide, proteins such as hyaluronic acid and chondroitin sulfate. celluloses such as hydroxymethyl cellulose, polyvinyl alcohol, and polyhydroxyalkyl (meth)acrylates.

Proteins and peptides may be covalently bound to the linker using conventional chemistries. Primary amine groups, such as found at the N-terminus or in lysine residues, will react with aldehydes and their equivalents under reductive conditions to give amines. (Molineux, Current pharmaceutical design, 10(11):1235-1244 (2004)). Mercapto (—SH) groups, such as found in cysteine residues, can undergo a conjugate addition with a variety of Michael acceptors, including acrylic and methacrylic acid derivatives, as well as maleimides (Gong et al., British Journal of Pharmacology, 163(2):399-412 (2011)). Other suitable nucleophilic groups found in peptides and proteins include disulfide bonds (Brocchini, et al., Nature protocols, 1:2241-2252 (2006)) and histidine residues (Cong, et al., Bioconjugate Chemistry, 23(2):248-263 (2012)).

The linker may be covalently joined to the protein or peptide using conventional chemistries. For instance, the linker polymer may be derivatized at one end with an electrophilic group such as an aldehyde, epoxide, halogen (chlorine, bromide, iodine), sulfonate ester (tosylate, mesylate), Michael acceptor, or activated carboxylates and then reacted with a nucleophilic amine or thiol group in the protein or peptide. Suitable Michael acceptors include acrylic and methacrylic acid derivatives such as acrylamides, methacrylamides, acrylates and methacrylates, as well as maleimides. Suitable activated carboxylates include nitrophenyl carbonate and NHS (N-hydroxy succinate) esters. In other embodiments, peptides and proteins containing arginine residues may be covalently joined with a linker containing a reactive 1,3 diketone functional group. The conjugates may be prepared by first joining the linker with the peptide or protein, followed by joining the linker with the branched poly(ethylene glycol), or by first joining the linker with the branched poly(ethylene glycol), followed by joining the linker with the peptide or protein. The optimal sequence of bond formation is determined by the specific chemical transformations involved.

In exemplified embodiments, PEG was selectively attached an N-terminus of TRAIL (WO 2007/145457, incorporated herein by reference). Such PEGylation reduced drug uptake and removal by hepatocytes and the hepatic reticuloendothelial system, leading to a decrease in TRAIL-mediated hepatoxicity. Additionally, PEGylation remarkably increased the solubility and stability of TRAIL (e.g., the stability, half-life and in vivo activity of PEGylated TRAIL was significantly greater than native-type TRAIL). Also, PEGylation was found to improve pharmacokinetic profiles of a linked drug with long-term storage in various formulations, thereby reducing drug administration frequencies and allowing sustained duration of effects of the drug. PEGylation is a gold standard to extend half-life of protein drugs and a highly efficient commercial strategy (Harris J M, and Chess R B. Nat Rev Drug Discov. 2003; 2(3):214-221, and Kang J S, et al., Expert Opin Emerg Drugs. 2009; 14(2):363-380). More than ten PEGylated biologics are FDA-approved (Alconcel SNS, et al., Polymer Chemistry. 2011; 2(7):1442-1448).

### TRAIL_{PEG}

TRAIL_{PEG}, a PEGylated trimeric TRAIL, is a lead compound that has been extensively investigated and has shown ability to reverse severe fibrosis in the liver and the pancreas of a subject by targeting activated hepatic and pancreatic stellate cells, respectively. TRAIL_{PEG} is a site-specifically PEGylated trimer isoleucine-zipper fusion human TRAIL. Bioengineered TRAIL with PEG improved its safety and pharmacokinetic profile in animals including monkeys. Kinase inhibitors utilized in this study are either FDA-approved or in clinical development.

### Macromolecules

In other embodiments, TRAIL can be derivatized as a long-acting TRAIL with an extended half-life using biopolymers or polypeptides through reported methods; for example, but not limited to, using chemically conjugated hyaluronic acid (Yang et al., Biomaterials 32(33); 8722-8729 (2011), depot forming polypeptides (Amiram et al., Proc natl Acad Sci USA, 110(8); 2792-2792 (2013), U.S. Published application Ser. No. 13/795,992) and TRAIL linked to extended recombinant polypeptides (U.S. Published application Ser. No. 12/699,761).

### Complexes

The TRAIL domain can be complexed with a negatively charged moiety. In some embodiments the negatively charged moiety can facilitate loading of the ligand or agonist into a nanoparticle for extended, sustained, or time released delivery. In some embodiments, the negatively charged moiety itself mediates extended, sustained, or time released delivery of the ligand or agonist.

The formation of a complex between positively charged TRAIL and the negatively charged chondroitin sulfate (CS) (CS/TRAIL) was developed and shown to facilitate loading of TRAIL in poly(lactide-co-glycolide) (PLGA) microspheres (MSs), without compromising the activity of the TRAIL (Kim, et al., Journal of Pharmacy and Pharmacology, 65(1):11-21 (2013). A nanocomplex of approximately 200 nm was formed in a weight ratio of 2 TRAIL to CS (TC2) at pH 5.0. The complex had >95% higher loading efficiency in PLGA MSs prepared by the multi-emulsion method than that of native TRAIL. Therefore, in some embodiments, the ligand or agonist, particularly TRAIL peptides, and variants, functional fragments and fusion proteins thereof, or conjugates thereof such as PEG-conjugates are complexed with chondroitin sulfate and optionally loaded into micro- or nanoparticles, for example, PLGA-based particles.

In other embodiments, the ligand or agonist, particularly TRAIL peptides, and variants, functional fragments and fusion proteins thereof, or conjugates thereof such as PEG-conjugates are complexed with hyaluronic acid (HA). Nanocomplexes of PEG-TRAIL and HA prepared by mixing positively charged PEG-TRAIL and negatively charged HA, were shown to have sustained delivery in vivo, with negligible loss of bioactivity compared with the PEG-TRAIL (Kim, et al., Biomaterials, 31(34):9057-64 (2010)). Delivery was further enhanced by administering the nanoparticles in a 1% HA containing solution. In an alternative embodiment, the HA is conjugated to the ligand or agonist as in Yang, et al., Biomaterials, 32(33):8722-9 (2011). Yang describes a coupling reaction between an aldehyde modified HA and the N-terminal group of IFNa, which can be used to couple HA to the pro-apoptotic agents disclosed herein. The IFNa content could be controlled in the range of 2-9 molecules per single HA chain with a bioconjugation efficiency higher than 95%, and the conjugates exhibited improved activity and half-life in vivo.

In some embodiments, the pro-apoptotic agent is modified to improve purification, Tag-removal, facilitate small molecule attachment or a combination thereof. Applied in tandem, elastin-like polypeptides and the Sortase A (SrtA) transpeptidase provide a method for chromatography-free purification of recombinant proteins and optional, site-specific conjugation of the protein to a small molecule (Bellucci, et al., Angewandte Chemie International Edition, 52(13):3703-3708 (2013)). This system provides an efficient mechanism for generating bioactive proteins at high yields and purities.

Other tags and labels are known in the art and include, for example, SUMO tags, His tags which typically include six or more, typically consecutive, histidine residues; FLAG tags, which typically include the sequence DYKDDDDK (SEQ ID NO:2); haemagglutinin (HA) for example, YPYDVP (SEQ ID NO:3); MYC tag for example ILKKATAYIL (SEQ ID NO:4) or EQKLISEEDL (SEQ ID NO:5). Methods of using purification tags to facilitate protein purification are known in the art and include, for example, a chromatography step wherein the tag reversibly binds to a chromatography resin.

Purification tags can be at the N-terminus or C-terminus of the fusion protein. The purification tags can be separated from the polypeptide of interest in vivo (e.g., during expression), or ex vivo after isolation of protein. Therefore, purification tags can also be used to remove the fusion protein from a cellular lysate following expression. The fusion protein can also include an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

Targeting Moieties

The TRAIL-conjugate, compositions including the TRAIL-conjugate agent, and delivery vehicles for the TRAIL-conjugate agent can include a targeting moiety. In some embodiments, the targeting moiety increases targeting to or accumulation of the pro-apoptotic agent to the organ of interest or target cells.

In one embodiment, the targeting moiety increases targeting to or accumulation of the pro-apoptotic agent to cancer cells, optionally in combination with KIs that are similarly targeted and/or co-formulated as targeted formulations.

In some embodiments, the targeting molecules are fused with or conjugated to the TRAIL-conjugate itself, or to a composition that includes the TRAIL-conjugate, or delivery vehicles carrying the TRAIL conjugate (e.g., a carrier such as a micro- or nanoparticle, liposome, etc).

The molecule can target a protein expressed in the cancer cells, or optionally on the surface of or in the microenvironment around targeted cancer cells. The targeting moiety can be, for example, an antibody or antibody fragment such as immunoglobulin (antibody) single variable domains (dAbs) that binds to an antigen expressed in an organ and/or tumor. In certain embodiments, the antibody is polyclonal, monoclonal, linear, humanized, chimeric or a fragment thereof. Representative antibody fragments are those fragments that bind the antibody binding portion of the non-viral vector and include Fab, Fab′, F(ab′), Fv diabodies, linear antibodies, single chain antibodies and bispecific antibodies known in the art. In certain embodiments, the targeting antibody or fragment thereof is specific for tumor cells.

### Formulations

Formulations of and pharmaceutical compositions including one or more active agents are provided. The pharmaceutical compositions can include one or more additional active agents. Therefore, in some embodiments, the pharmaceutical composition includes two, three, or more active agents. The pharmaceutical compositions can be formulated as a pharmaceutical dosage unit, also referred to as a unit dosage form. Such formulations typically include an effective amount a TRAIL-conjugate. Effective amounts of the disclosed TRAIL-conjugates are discussed in more detail below.

Pharmaceutical compositions can be for administration by parenteral (intramuscular, intraperitoneal, intravenous (IV) or subcutaneous injection), or nasal or pulmonary administration and can be formulated in dosage forms appropriate for each route of administration.

Optionally, the compositions are administered locally, for example by injection directly into a site to be treated (e.g., into a tumor). In some embodiments, the compositions are injected or otherwise administered directly into the vasculature at or adjacent to the intended site of treatment (e.g., adjacent to a tumor). Typically, local administration causes an increased localized concentration of the compositions which is greater than that which can be achieved by systemic administration.

The formulations are optionally an aqueous solution, a suspension or emulsion. Such compositions include diluents sterile water, buffered saline of various buffer content (e.g., Tris-HCl, acetate, phosphate), pH and ionic strength; and optionally, additives such as detergents and solubilizing agents (e.g., TWEEN™20, TWEEN™80 also referred to as polysorbate 20 or 80. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### Building Potent Anticancer Drugs With Negligible Side Effects

Current combination chemotherapies offer moderate efficacy with a major challenge—a broad range of side effects. Although such agents provide valuable options, they demonstrate numerous, partly severe side effects that can eventually involve a fatal outcome. Therefore, introducing a potent therapeutic approach with significantly reduced side effects and wide applicability to diverse types of cancers is a long-felt need in the field. Besides so-called “conventional” chemotherapies, molecularly-targeted agents such as kinase inhibitors (KIs) and antibodies targeting growth factor signaling pathways have been introduced with the initial expectation of substantially reduced side effects (Noble M E, et al., Science. 2004; 303(5665):1800-1805, Eckstein N, et al., J Exp Clin Cancer Res. 2014; 33:15, and Hansel T T, et al., Nat Rev Drug Discov. 2010; 9(4):325-338).

### Kinase Inhibitors

Tyrosine kinases are a class of enzymes that catalyze the transfer of the terminal phosphate of adenosine triphosphate to tyrosine residues in protein substrates. Tyrosine kinases are believed, by way of substrate phosphorylation, to play critical roles in signal transduction for a number of cell functions. Though the exact mechanisms of signal transduction is still unclear, tyrosine kinases have been shown to be important contributing factors in cell proliferation, carcinogenesis and cell differentiation. Tyrosine kinases can be categorized as receptor type or non-receptor type. Receptor type tyrosine kinases have an extracellular, a transmembrane, and an intracellular portion, while non-receptor type tyrosine kinases are wholly intracellular. Select kinase inhibitors (especially for efficacy in HT-29, MDA-MD-231, A549, LNCAP, HP LNCAP, DU-145, PC3 and other cells) include A-674563, Afatinib (BIBW2992), Apatinib, AST-1306, AT7519, AT9283, AZ 960, AZD3463, AZD5438, BGJ398, BMS-265246, Bosutinib, Canertinib, CCT137690, CHIR-124, CHIR-98014, CP-673451, CYT387, Dacomitinib, Dactolisib, Dasatinib, Dinaciclib, Dovitinib, ENMD-2076, Flavopiridol HCl, Foretinib, GSK1904529A, Idelalisib, INCB28060, Lapatinib, , Lenvatinib, Linifanib, Linsitinib, LY2784544, MGCD-265, Milciclib, Neratinib, OSI-930, Pazopanib, PD168393, PD98059, Pelitinib, PF-00562271, PHA-767491, PHA-793887, PIK-75, Regorafenib, Seliciclib, Saracatinib, SGX-523, SNS-032, Sunitinib Malate, TAK-901, TG101209, Tyrphostin, U0126-EtOH, Volasertib, WZ4002 and ZM 306416. These Ms target the following kinases: anaplastic lymphoma kinase (ALK), fms-like tyrosine kinase 3 (FLT3), vascular endothelial growth factor receptor (VEGFR), Bcr-Abl, CD117 (c-Kit), Src, cyclin-dependent kinase (CDK), colony stimulating factor 1 receptor (CSF-1R), c-met, C-met, platelet-derived growth factor receptor (PDGFR), epidermal growth factor receptor (EGFR), human epidermal growth factor receptor 2 (HER2), focal adhesion kinase (FAK), fibroblast growth factor receptor (FGFR), glycogen synthase kinase 3 (GSK-3), insulin-like growth factor 1 receptor (IGF-1R), Janus kinase (JAK), mitogen-activated protein kinase kinase (MEK), phosphoinositide 3-kinase (PI3K), mammalian target of rapamycin (mTOR), ATM/ATR, Akt, DNA-dependent protein kinase (DNA-PK) and Tie-2. Other exemplary kinase inhibitors include nintedanib, brivanib, cediranib, masitinib, orantinib and ponatinib.

Solid tumors can be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer. Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes. As detailed herein, a method of employing KIs to sensitize tumor cells to TRAIL-based agents for targeted cancer therapy has been newly identified.

### Kits

The invention also includes kits that include a composition of the invention, optionally also including a compound (e.g. KI inhibitor and TRAIL_{PEG}), and instructions for use.

### Pharmaceutical Compositions

Another aspect of the invention pertains to pharmaceutical compositions of the compounds of the invention. The pharmaceutical compositions of the invention typically comprise a compound of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds can be administered in a time release formulation, for example in a composition which includes a slow release polymer, or in a fat pad described herein. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, certain methods of preparation are vacuum drying and freeze-drying which yields a powder of the active ingredient plus any additional desired ingredient from a previously sterile-filtered solution thereof

Depending on the route of administration, the compound may be coated in a material to protect it from the action of enzymes, acids and other natural conditions which may inactivate the agent. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes (Strejan, et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., KI and TRAIL_{PEG}) preferably is formulated in the composition in a therapeutically effective amount. A “therapeutically effective amount” refers to an amount effective, at dosages and for periods of time necessary, to achieve the desired therapeutic result to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Exemplary dosages of compounds (e.g., KI and/or TRAIL_{PEG}) of the invention include e.g., about 0.0001% to 5%, about 0.0001% to 1%, about 0.0001% to 0.1%, about 0.001% to 0.1%, about 0.005%-0.1%, about 0.01% to 0.1%, about 0.01% to 0.05% and about 0.05% to 0.1%.

Exemplary dosages for oral KIs can range from about 1 mg to 1 g, including about 20 mg to 1 g, about 50 mg to 1 g, about 75 mg to 1 g, about 100 mg to over 800 mg (e.g., 900 mg, 1 g, 1.5 g, 2 g or more). For example, dasatinib—100 mg or 140 mg daily; regorafenib—160 mg daily; bosutinib—500 mg daily; pazopanib—800 mg daily.

The compound(s) of the invention can be administered in a manner that prolongs the duration of the bioavailability of the compound(s), increases the duration of action of the compound(s) and the release time frame of the compound by an amount selected from the group consisting of at least 3 hours, at least 6 hours, at least 12 hours, at least 24 hours, at least 48 hours, at least 72 hours, at least 4 days, at least 5 days, at least 6 days, at least 7 days, at least 2 weeks, at least 3 weeks, and at least a month, but at least some amount over that of the compound(s) in the absence of the fat pad delivery system. Optionally, the duration of any or all of the preceding effects is extended by at least 30 minutes, at least an hour, at least 2 hours, at least 3 hours, at least 6 hours, at least 12 hours, at least 24 hours, at least 48 hours, at least 72 hours, at least 4 days, at least 5 days, at least 6 days, at least 7 days, at least 2 weeks, at least 3 weeks or at least a month.

A compound of the invention can be formulated into a pharmaceutical composition wherein the compound is the only active agent therein. Alternatively, the pharmaceutical composition can contain additional active agents. For example, two or more compounds of the invention may be used in combination. Moreover, a compound of the invention can be combined with one or more other agents that have modulatory effects on cancer. One specific discovery of the invention is the instant identification of a combinatorial treatment that employs both KIs and long-acting TRAIL-based agonists.

This invention is further illustrated by the following examples which should not be construed as limiting. The contents of all references, patents, and published patent applications cited throughout this application, as well as the figures, are incorporated herein by reference.

### EXAMPLES

### Example 1

### Kinase inhibitor (KI) screen: select KI sensitize TRAIL resistant colorectal cancer cells to TRAIL_{PEG}-induced apoptosis

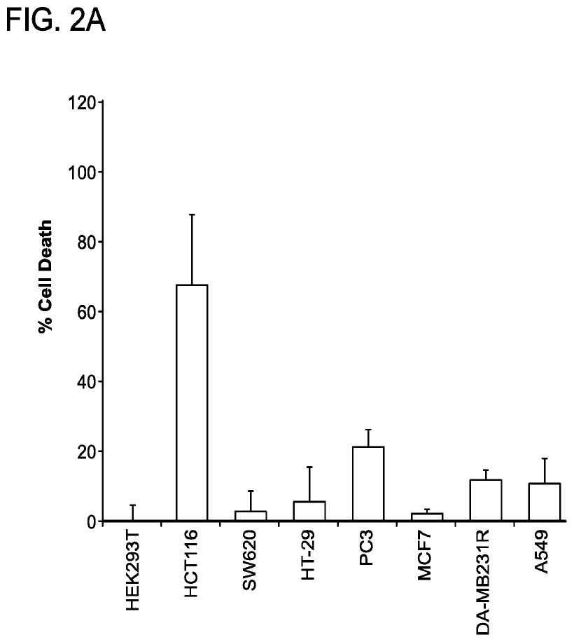

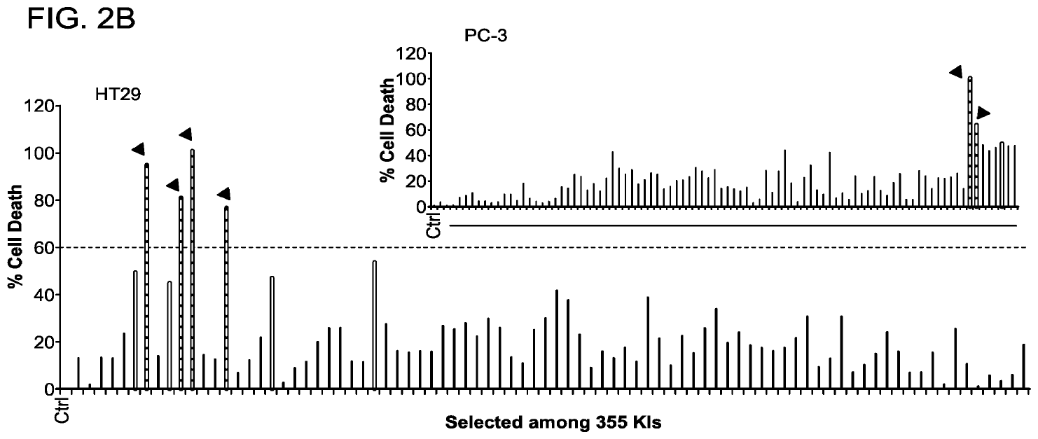

A library of KIs was screened for TRAIL sensitization in various TRAIL-resistant cancer cells, including: HT29 (CRC), MDA-MB-231 (breast), LNCAP (prostate), DU145 (prostate), PC3 (prostate) and A549 (lung). TRAIL-PEG (1 μg/mL) alone failed to induce effective cell death when administered to these cells (FIG. 2A). In contrast, when HT29 CRC cells were pretreated with a diverse set of 355 KIs (Selleckhem, Houston) for 24 hours before TRAIL_{PEG} treatment, KI pretreatment substantially increased TRAIL_{PEG}-induced cell death and apoptosis, as confirmed by both cell death assays and Western blot analysis. The 355 KIs comprised of compounds targeting diverse kinases, including multi kinases, RTK (receptor kinase tyrosine), PI3K (phosphinistide 3-kinase), aurora kinases, including multi (mitogen-activated protein kinase). In these initial screening studies, about 11 LIs—OSI-930, saracatinib (AZD0530), ENMD-2076, PD98059, U0126-EtOH, Idelalisib (CAL-101, GS-1101), dactolisib (BEZ235), regorafenib (BAY 73-4506, Stivarga), dasatinib (BMS-354825, Sprycel), pazopanib (Votrient) and bosutinib (SKI-606, Bosulif)—demonstrated synergistic efficacy when combined with TRAIL_{PEG} (FIG. 2B). FIGS. 2A-2B show relative cell death rates determined by the ratio (M+TRAIL_{PEG})/(KI alone) after two separate cell death assays, where increased cell death purely from combined M and TRAIL_{PEG} was demonstrated. The interaction between KIs and TRAIL had been previously explored in vitro with the tyrosine KI sorafenib, a drug similar to regorafenib. However, such studies were mostly performed on cellular levels and not in in vivo models, combined with systemically administered recombinant TRAIL. Although a similar structure, regorafenib was newly approved in 2012. The interactions between the three other selected KIs and TRAIL have not been previously reported, in vitro or in vivo.

The results indicated that TRAIL-resistant cancer cells became highly sensitive to TRAIL-based agents when pretreated with select Ms. A few KIs significantly improved TRAIL-mediated apoptosis in certain types of cancer cells (Table 1). Additional details are described in subsequent examples.

TABLE 1
Example KIs that sensitize cancer cells to TRAIL-based agents.

| Human Cancer Cell | KI | KI Target |
|---|---|---|
| Line | | |
| HT-29 | OSI-930 | c-Kit, CSF-1R, |
| | | VEGFR |
| colorectal | Pazopanib | PDGFR, c-Kit, |
| | | VEGFR |
| adenocarcinoma | Saracatinib (AZD0530) | Src, Bcr-Abl |
| | Bosutinib (SKI-606) | Src |
| | Dasatinib | Bcr-Abl, c-Kit, |
| | | Src |
| | Regorafenib (BAY 73-4506) | c-RET, VEGFR |
| | ENMD-2076 | Aurora Kinase, |
| | | FLT3, VEGFR |
| | PD98059 | MEK |
| | U0126-EtOH | MEK |
| | CAL-101 (Idelalisib, | PI3K |
| | GS-1101) | |
| | BEZ235 (NVP-BEZ235, | PI3K, ATM/ATR, |
| | Dactolisib) | mTOR |
| MDA-MB-231 | Pelitinib (EKB-569) | EGFR |
| breast cancer | AT9283 | JAK, Aurora |
| | | Kinase, Bcr-Abl |
| | Dasatinib | Bcr-Abl, c-Kit, |
| | | Src |
| | Canertinib (CI-1033) | EGFR, HER2 |
| | PHA-793887 | CDK |
| | Roscovitine (Seliciclib, | CDK |
| | CYC202) | |
| | SNS-032 (BMS-387032) | CDK |
| | PIK-75 | PI3K, DNA-PK |
| | LY2784544 | JAK |
| | PF-00562271 | FAK |
| | AZ 960 | JAK |
| | CYT387 | JAK |
| | Volasertib (BI 6727) | PLK |
| | A-674563 | PKA, CDK, Akt |
| | Flavopiridol HCl | CDK |
| | TG101209 | JAK, FLT3, c-RET |
| | TAK-901 | Aurora Kinase |
| | BMS-265246 | CDK |
| | CHIR-124 | Chk |
| | Dacomitinib (PF299804, | EGFR |
| | PF299) | |
| | PHA-767491 | CDK |
| | CCT137690 | Aurora Kinase |
| | CHIR-98014 | GSK-3 |
| | Milciclib (PHA-848125) | CDK |
| | Dinaciclib (SCH727965) | CDK |
| | Dovitinib (TKI-258) | PDGFR, FGFR, |
| | Dilactic Acid | c-Kit, FLT3, |
| | | VEGFR |
| A549 | WZ4002 | EGFR |
| Lung carcinoma | AT7519 | CDK |
| | SNS-032 (BMS-387032) | CDK |
| | GSK1904529A | IGF-1R |
| | Linifanib (ABT-869) | CSF-1R, PDGFR, |
| | | VEGFR |
| | Afatinib (BIBW2992) | EGFR, HER2 |
| | Lapatinib (GW-572016) | HER2, EGFR |
| | Ditosylate | |
| | Apatinib | VEGFR |
| | AZD5438 | CDK |
| | Flavopiridol HCI | CDK |
| | CP-673451 | PDGFR |
| | BMS-265246 | CDK |
| | BGJ398 (NVP-BGJ398) | FGFR |
| | CHIR-124 | Chk |
| | Dinaciclib (SCH727965) | CDK |
| LNCAP | OSI-906 (Linsitinib) | IGF-1R |
| prostate | Sunitinib Malate | VEGFR, PDGFR, |
| | | c-Kit |
| adenocarcinoma | SGX-523 | c-Met |
| | Afatinib (BIBW2992) | EGFR, HER2 |
| | Lapatinib (GW-572016) | HER2, EGFR |
| | Ditosylate | |
| | INCB28060 | c-Met |
| | Tyrphostin 9 | EGFR |
| | ZM 306416 | VEGFR |
| | Tyrphostin AG 1296 | FGFR, c-Kit, |
| | | PDGFR |
| HP LNCAP | WZ4002 | EGFR |
| prostate | MGCD-265 | Tie-2, VEGFR, |
| | | c-Met |
| adenocarcinoma | Regorafenib (BAY 73-4506) | c-RET, VEGFR |
| | SGX-523 | c-Met |
| | Lenvatinib (E7080) | VEGFR |
| | Tyrphostin AG 879 | HER2 |
| | Tyrphostin 9 | EGFR |
| | PD168393 | EGFR |
| DU-145 | Neratinib (HKI-272) | HER2, EGFR |
| prostate | Afatinib (BIBW2992) | EGFR, HER2 |
| adenocarcinoma | Foretinib (GSK1363089) | VEGFR, c-Met |
| | AST-1306 | EGFR |
| | Dacomitinib (PF299804, | EGFR |
| | PF299) | |
| | AZD3463 | ALK |
| | Tyrphostin 9 | EGFR |
| PC3 | Regorafenib (BAY 73-4506) | c-RET, VEGFR |
| prostate | CP-673451 | PDGFR |
| adenocarcinoma | AST-1306 | EGFR |
| | Tyrphostin 9 | EGFR |
| | AZD3463 | ALK |

### Selection of Multi-Targeted KIs to Sensitize TRAIL-Resistant HT29 CRC Cells to TRAIL_{PEG}-Induced Apoptosis (Through Caspase Activation and Downregulating Anti-Apoptotic Markers)

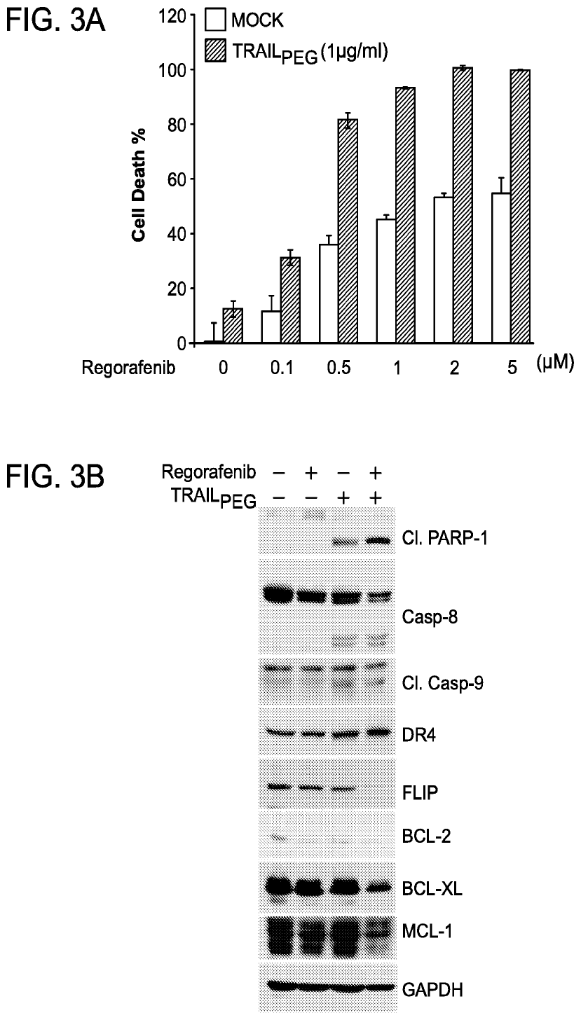

Regorafenib potentiated TRAIL-induced apoptosis in HT-29 cells when combined with TRAIL_{PEG} (1 μg/mL) (FIG. 3A). After treating cells with regorafenib (2 μM), caspases and anti-apoptotic proteins were analyzed by western blotting (FIG. 3B). Regorafenib significantly sensitized caspase-dependent TRAIL_{PEG} -induced apoptosis, as evidenced by PARP-1 cleavage and caspase activation as well as downregulated anti-apoptotic proteins, c-FLIP, MCL-1, BCL-2, and BCL-XL. Regorafenib also dose-dependently downregulated RIP-1, a molecule associated with NF-κB (a cell survival pathway), which implied that sensitization mechanisms by this compound were also associated with inhibiting a TRAIL-induced cell survival pathway. Data confirmed that FDA-approved KIs or KIs under clinical development synergized with long-acting TRAIL_{PEG} by overcoming TRAIL-resistance through unique TRAIL sensitization mechanisms.

A unique class of KIs that sensitized breast, prostate and lung cancer cells against TRAIL-induced apoptosis had therefore been discovered. The role of KIs on TRAIL sensitization in other cancer cells was further extrapolated, with implications for the development of KI/TRAIL_{PEG} combinations as universal anticancer agents. It was contemplated that select KIs could be used with TRAIL_{PEG} for clinical therapy and imaging: in particular, (1) M can be used as a relatively less toxic and patient-friendly (orally active) TRAIL sensitizer for anticancer therapy with TRAIL_{PEG} and (2) a biomarker of TRAIL sensitization (e.g. DR or caspase-3) can be employed as a noninvasive molecular imaging tool.

### Selection of KIs Utilizing Cell-Based Screening

A few Ms, such as dasatibin, pazopanib, and bosutinib, were newly discovered as TRAIL sensitizers for CRC via screening in HT29 cells, therefore other KIs in other CRC cells are identified. A library of KIs is assessed to identify the TRAIL-sensitizing ability of component KIs (e.g., a Selleckchem M library, comprised of 355 KIs dissolved in DMSO to a final concentration of 10 mM is employed). The screening is performed using an MTT cell death assay in CRC cells with different TRAIL sensitivities. Cell lines that are tested include TRAIL-sensitive cells (e.g., HCT116 and SW480), and TRAIL-resistant cells (e.g., HT29 and SW620), human colon fibroblasts (e.g., CCD-18Co), and primary tumor cells from CRC patients.

The dose-dependent toxic effects of the KIs is examined after a 24 hour incubation with the cells at four doses of KIs (e.g., 0.1-5 μM). Next, the enhanced TRAIL_{PEG} effect on CRC cell death in the presence of each M is investigated at optimized TRAIL_{PEG} concentration ranges (e.g., 0-15 μM, or 0-1 μg/mL). Synergistic effects of the combined modalities are evaluated using combination index analysis. Selected compounds (e.g. four compounds in the case of HT29 cells and the 355 KI library) show superior synergism with TRAIL_{PEG} against all tested CRC (or other cancer) cells.

### Example 2

### Role of Selected Kinases on TRAIL-Sensitizing Mechanisms in CRC Cells

### A Multi-Targeted M Induced DR4 While Downregulating Dcr2 and Anti-Apoptotic Proteins

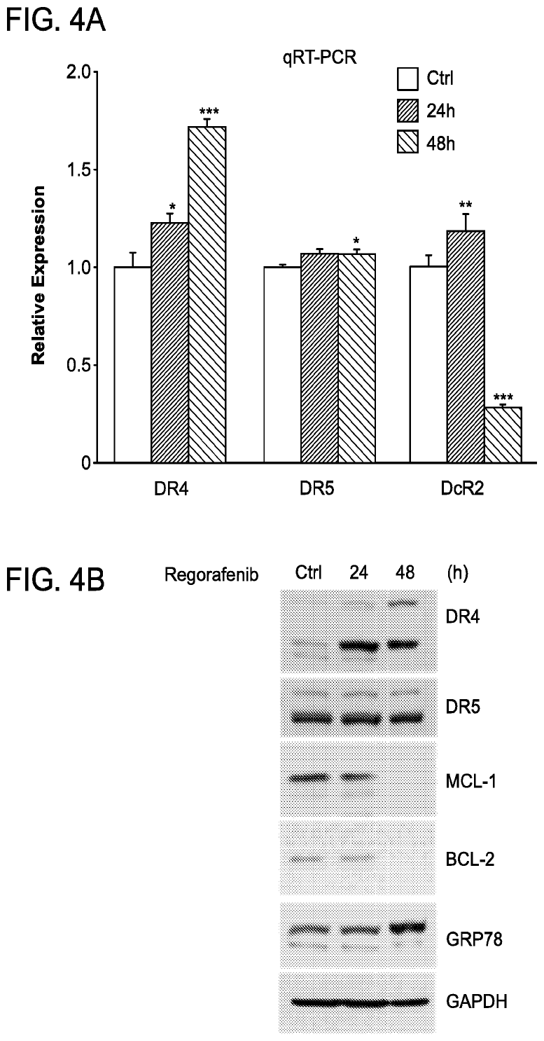

TRAIL signaling is complex, and multiple mechanisms are involved in TRAIL resistance and sensitization. Malfunction of TRAIL receptors, e.g., defects in the expression and/or localization of DR4/DR5 at the cell surface or increased expression of decoy receptors, DcR1/DcR2, often results in TRAIL resistance in cancer cells. Regorafenib was identified to significantly upregulate DR4 while down-regulating DcR2 in HT29 cells (FIGS. 4A and 4B), thus increasing TRAIL-induced apoptosis.

HT29 cells were treated with regorafenib (2 μM) for 24 hour or 48 hour as indicated. mRNAs for TRAIL receptors were measured by quantitative RT-PCR (qPCR) (FIG. 4A). Regorafenib-treated CRC cells upregulated DR4, but minimally induced DRS. The expression of DcR2, decoy receptor functioning as a TRAIL signaling competitor, was rarely detectable on cells treated for 48 hour. After treating HT29 cells with regorafenib as indicated, DR4/5 or anti-apoptotic proteins were analyzed by Western blot (FIG. 4B). Regorafenib-treated CRC cells highly expressed DR4 protein, consistent with the qPCR results (FIG. 4A). Conversely, anti-apoptotic proteins MCL-1 and BCL-2 were absent in HT29 cells treated with regorafenib for 48 hours. It has been previously reported that TRAIL receptors could be induced by signaling including NF-κB, ER stress, and JNK-ROS. The expression of GRP78, a representative biomarker of ER stress, also showed a similar pattern as that observed for DR4.

### Example 3

### Selection of Multi-Targeted KIs to Sensitize TRAIL-Resistant Prostate Cancer Cells to TRAIL_{PEG}-Induced Apoptosis (Through Caspase Activation and Downregulating Anti-Apoptotic Markers)

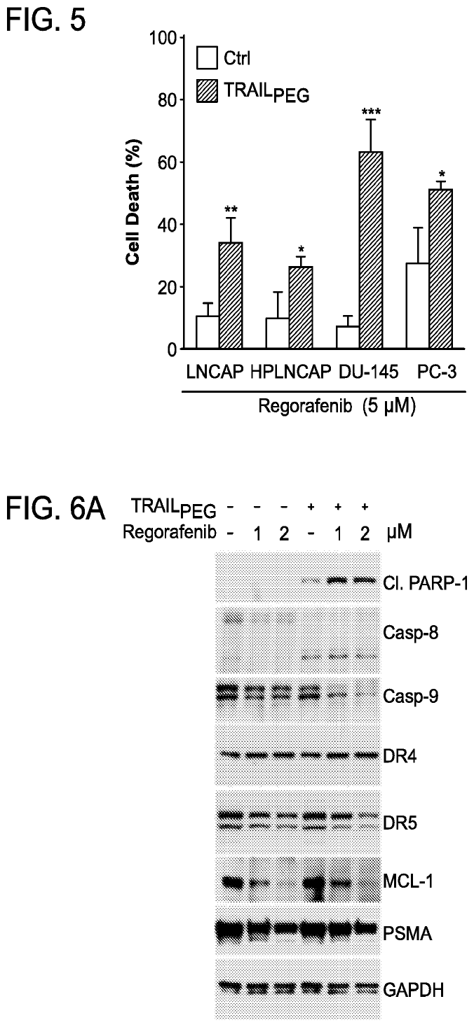

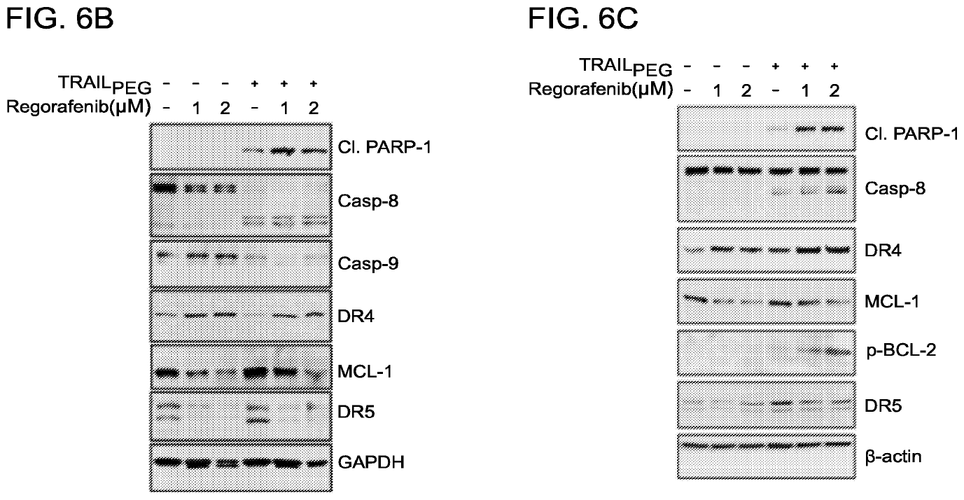

Regorafenib potentiated TRAIL-induced apoptosis in prostate cancer cells (LNCAP, HPLNCAP (High Passages LNCAP), DU-145 and PC-3) when combined with TRAIL_{PEG} (1 μg/mL) (FIG. 5). After treating cells with regorafenib (5 μM), caspases and anti-apoptotic proteins were analyzed by western blotting (FIG. 6A-FIG. 6C). Regorafenib or TRAIL_{PEG} alone did not induce strong apoptosis in tested prostate cancer cells. In contrast, when combined, regorafenib significantly sensitized caspase-dependent TRAIL_{PEG} -induced apoptosis, as evidenced by PARP-1 cleavage and caspase activation as well as downregulated anti-apoptotic proteins, MCL-1. Data confirmed that FDA-approved KIs or KIs under clinical development synergized with long-acting TRAIL_{PEG} by overcoming TRAIL-resistance through unique TRAIL sensitization mechanisms.

### Example 4

### Determination of the Anticancer Efficacy and Safety of Oral KIs for TRAIL-Based Cancer Therapy

An orally administered selected M combined with systemic TRAIL_{PEG} possessing extended half-life (e.g., long-acting) is contemplated to demonstrate superior efficacy in CRC in vivo, with reduced systemic toxicity. Potentiated TRAIL-induced apoptosis in vivo is a result of M-induced TRAIL sensitizing, as is demonstrated in vivo.

The efficacy of KI/TRAIL_{PEG} and selected oral KIs is evaluated in tumor xenograft bearing TRAIL-sensitive/resistant cells, as well as in primary CRC cells, identifying KI/TRAIL_{PEG} as a potent anticancer drug while noninvasively monitoring DR regulation and apoptosis activities via molecular imaging. The efficacy of KI/TRAIL_{PEG} is demonstrated in multiple CRC models to address genomic heterogeneity of CRC.

The KI/TRAIL_{PEG} combo is evaluated in various CRC tumors possessing different TRAIL sensitivities in vivo with improved safety profiles. After in vivo studies, an in-depth analysis is performed by analyzing various markers described from tumor tissues isolated from xenograft models. Tissues and blood samples are analyzed for biomarkers. Representative biomarkers identified in such studies are screened in CRC tissues and normal colon tissues obtained from patients, to predict sensitivity of such CRC tissues to TRAIL-based therapies in the clinic.

### Orally Delivered Regorafenib Combined With Systemic TRAIL_{PEG} Induced DR-Mediated Apoptosis and Auppressed TRAIL Resistant HT29 Tumor Growth In Vivo

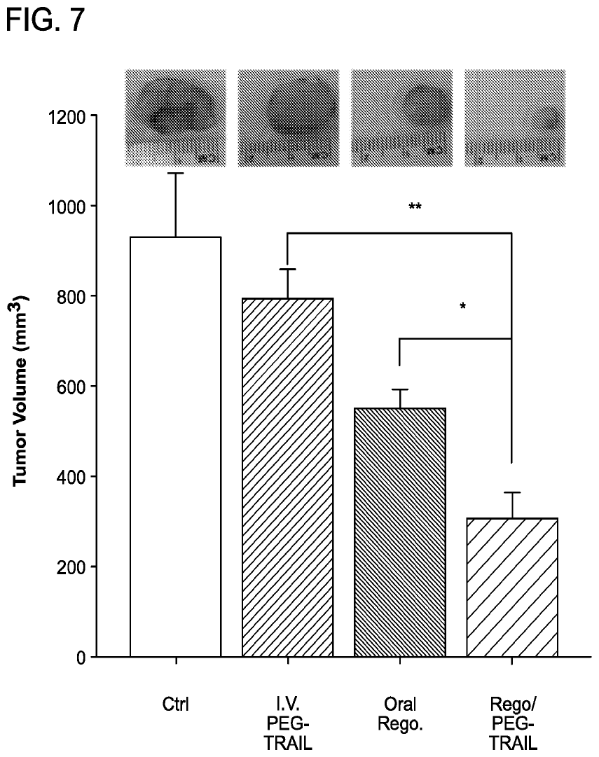

The therapeutic combination of oral regorafenib and TRAIL_{PEG} in HT29 xenografts in comparison to regorafenib and TRAIL_{PEG} alone (FIG. 7) were investigated. HT29 xenografts were treated with oral regorafenib (10 mg/kg) or saline on the 12th, 14th, and 16th days of tumor inoculation. On the 13th, 15th, and 17th days, animals were given an i.v. dose of TRAIL_{PEG} (150 μg). Animals were sacrificed on day 27. TRAIL_{PEG} did not show the efficacy that it did in TRAIL-resistant cells. Regorafenib demonstrated a moderate tumor reduction after three non-daily doses. In contrast, the combination of regorafenib/TRAIL_{PEG} therapy suppressed tumor growth significantly, as compared to drug alone, with no observed adverse effects. Unlike chemotoxic drugs like DOX, which sensitized tumors only at highly toxic doses near-maximum tolerated dose (MTD), regorafenib showed synergism with TRAIL_{PEG}, without significant toxicity and at a lower dose (regorafenib's MTD=160 mg/kg) (Strumberg D, Br J Cancer. 2012; 106(11):1722-1727).

Taken together with the results from the HT29 xenografts and human colon tumor tissues, molecularly-targeting CRC through oral KI and TRAIL_{PEG}, an efficient therapy is demonstrated in preclinical models and particularly in cancer patients. Mechanisms of KI/TRAIL_{PEG} on in vivo TRAIL signaling in CRC tumors are explored as described herein.

### Equivalents

Those skilled in the art will recognize, or be able to ascertain using no more than routine experimentation, many equivalents to the specific embodiments of the invention described herein. Such equivalents are intended to be encompassed by the following claims.

1. A method for sensitizing a cancer of a subject to treatment with a death receptor agonist, the method comprising:
administering a kinase inhibitor (KI) to the subject in an amount sufficient to sensitize the cancer to treatment with a long-acting death receptor agonist,
thereby sensitizing the cancer of the subject to treatment with a death receptor agonist.

2. The method of claim 1, wherein the KI is selected from the group consisting of A-674563, Afatinib (BIBW2992), Apatinib, AST-1306, AT7519, AT9283, AZ 960, AZD3463, AZD5438, BGJ398, BMS-265246, Bosutinib, Canertinib, CCT137690, CHIR-124, CHIR-98014, CP-673451, CYT387, Dacomitinib, Dactolisib, Dasatinib, Dinaciclib, Dovitinib, ENMD-2076, Flavopiridol HCl, Foretinib, GSK1904529A, Idelalisib, INCB28060, Lapatinib, Lenvatinib, Linifanib, Linsitinib, LY2784544, MGCD-265, Milciclib, Neratinib, OS-930, Pazopanib, PD168393, PD98059, Pelitinib, PF-00562271, PHA-767491, PHA-793887, PIK-75, Regorafenib, Seliciclib, Saracatinib, SGX-523, SNS-032, Sunitinib Malate, TAK-901, TG101209, Tyrphostin, U0126-EtOH, Volasertib, WZ4002 and ZM 306416

3. The method of claim 1, wherein the KI target is selected from the group consisting of VEGFR, Src, MEK, PI3K, EGFR, CDK, JAK, CDK and c-Met.

4. The method of claim 1, wherein the KI is orally administered, optionally at a dosage of 1 mg to 1 g per tablet.

5. The method of claim 1, wherein the cancer is selected from the group consisting of sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer including prostate adenocarcinoma, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, retinoblastoma, multiple myeloma, rectal carcinoma, thyroid cancer, head and neck cancer, brain cancer, cancer of the peripheral nervous system, cancer of the central nervous system, neuroblastoma, colorectal adenocarcinoma and cancer of the endometrium.

6. The method of claim 1, wherein the cancer is a tumor necrosis factor-related apoptosis inducing ligand (TRAIL)-resistant cancer.

7. The method of claim 1, further comprising administering a long-acting death receptor agonist to the subject.

8. The method of claim 7, wherein the death receptor agonist is systemically administered.

9. The method of claim 7, wherein the death receptor agonist and the KI are co-administered.

10. The method of claim 1, wherein the death receptor agonist comprises a tumor necrosis factor (TNF)-related apoptosis-inducing ligand (TRAIL), a TRAIL analogue, death receptor agonist antibodies, or a derivative thereof.

11. The method of claim 1, wherein the death receptor agonist comprises human recombinant TRAIL, a human TRAIL analogue, or a derivative thereof.

12. The method of claim 1, wherein the death receptor agonist comprises native TRAIL, a native TRAIL analogue, or a derivative thereof.

13. The method of claim 1, wherein the death receptor agonist is selectively attached to a polymer.

14. The method of claim 13, wherein the polymer comprises polyethylene glycol (PEG), or derivative thereof.

15. The method of claim 14, wherein the PEG is selected from the group consisting of methoxypolyethylene glcycol succinimidyl propionate, methoxypolyethylene glycol succinate N-hydroxysuccinimide, methoxypolyethylene glycol propionaldehyde, methoxypolyethylene glycol maleimide, and multiple-branched polyethylene glycol.

16. The method of claim 1, wherein the death receptor agonist comprises PEGylated trimeric isoleucine-zipper fused TRAIL (TRAILPEG).

17. The method of claim 1, wherein the cancer is colorectal cancer.

18. The method of claim 1, wherein the KI is selected from the group consisting of OSI-930, Pazopanib, Saracatinib (AZD0530), Bosutinib (SKI-606), Dasatinib, Regorafenib (BAY 73-4506), ENMD-2076, PD98059, U0126-EtOH, CAL-101 (Idelalisib, GS-1101) and BEZ235 (NVP-BEZ235, Dactolisib) and the cancer is colorectal adenocarcinoma.

19. The method of claim 1, wherein the KI is selected from the group consisting of Pelitinib (EKB-569), AT9283, Dasatinib, Canertinib (CI-1033), PHA-793887, Roscovitine (Seliciclib,CYC202), SNS-032 (BMS-387032), PIK-75, LY2784544, PF-00562271, AZ 960, CYT387, Volasertib (BI 6727), A-674563, Flavopiridol HCl, TG101209, TAK-901, BMS-265246, CHIR-124, Dacomitinib (PF299804, PF299), PHA-767491, CCT137690, CHIR-98014, Milciclib (PHA-848125), Dinaciclib (SCH727965) and Dovitinib (TKI-258) Dilactic Acid and the cancer is breast cancer.

20. The method of claim 1, wherein the KI is selected from the group consisting of WZ4002, AT7519, SNS-032 (BMS-387032), GSK1904529A, Linifanib (ABT-869), Afatinib (BIBW2992), Lapatinib (GW-572016) Ditosylate, Apatinib, AZD5438, Flavopiridol HCl, CP-673451, BMS-265246, BGJ398 (NVP-BGJ398), CHIR-124 and Dinaciclib (SCH727965) and the cancer is lung cancer.

21. The method of claim 1, wherein the KI is selected from the group consisting of Afatinib (BIBW2992), AST-1306, AZD3463, CP-673451, Dacomitinib (PF299804, PF299), Foretinib (GSK1363089), INCB28060, Lapatinib (GW-572016) Ditosylate, Lenvatinib (E7080), MGCD-265, Neratinib (HKI-272), OSI-906 (Linsitinib), PD168393, Regorafenib (BAY 73-4506), SGX-523, Sunitinib Malate, Tyrphostin 9, Tyrphostin AG 1296, Tyrphostin AG 879, WZ4002 and ZM 306416 and the cancer is prostate adenocarcinoma.

22. (canceled)

23. (canceled)

24. A method for identifying a kinase inhibitor (KI) capable of sensitizing a cancer cell to a death receptor agonist comprising:
contacting the cancer cell with a KI;
contacting the cancer cell with a death receptor agonist; and
detecting cell death or a marker of apoptosis in the cancer cell administered the KI, as compared to an appropriate control cell,
thereby identifying a kinase inhibitor (KI) capable of sensitizing a cancer cell to a death receptor agonist.

25. The method of claim 24, wherein the cancer cell is contacted with the KI for at least 3 hours, optionally for 6 hours or more, 12 hours or more, or 24 hours or more, in advance of contacting the cancer cell with the death receptor agonist.

26. The method of claim 24, wherein cell death or a marker of apoptosis in the cancer cell is measured by a cell death assay, an imaging agent, or by Western blot.

27. The method of claim 24, wherein the KI is selected from the group consisting of A-674563, Afatinib (BIBW2992), Apatinib, AST-1306, AT7519, AT9283, AZ 960, AZD3463, AZD5438, BGJ398, BMS-265246, Bosutinib, Canertinib, CCT137690, CHIR-124, CHIR-98014, CP-673451, CYT387, Dacomitinib, Dactolisib, Dasatinib, Dinaciclib, Dovitinib, ENMD-2076, Flavopiridol HCl, Foretinib, GSK1904529A, Idelalisib, INCB28060, Lapatinib, Lenvatinib, Linifanib, Linsitinib, LY2784544, MGCD-265, Milciclib, Neratinib, OSI-930, Pazopanib, PD168393, PD98059, Pelitinib, PF-00562271, PHA-767491, PHA-793887, PIK-75, Regorafenib, Seliciclib, Saracatinib, SGX-523, SNS-032, Sunitinib Malate, TAK-901, TG101209, Tyrphostin, U0126-EtOH, Volasertib, WZ4002 and ZM 306416.

28. A method for treating or preventing a cancer in a subject, the method comprising:
administering a kinase inhibitor and a death receptor agonist to the subject in an amount sufficient to treat or prevent the cancer in the subject,
thereby treating or preventing the cancer in the subject.

29. The method of claim 28, wherein the KI and the death receptor agonist act synergistically to treat or prevent the cancer in the subject.
